(19) United States
(12) Patent Application Publication
(10) Pub. No.: US 2010/0062994 A1
(43) Pub. Date: Mar. 11, 2010

(54) Adenosine Receptor Antagonists

(75) Inventors: Jacob Ravn, Skovlunde (DK); Christoph Rosenbohm, Birkerod (DK); Katrine Qvortrup, Soborg (DK); Troels Koch, Copenhagen S (DK)

Correspondence Address: STERNE, KESSLER, GOLDSTEIN & FOX P.L.L.C., 1100 NEW YORK AVENUE, N.W., WASHINGTON, DC, 20005, US

(73) Assignee: SANTARIS PHARMA A/S, HØRSHOLM (DK)

(21) Appl. No.: 12/373,640

(22) PCT Filed: Jul. 6, 2007
(86) PCT No.: PCT/DK07/00345

§ 371 Date: Aug. 28, 2009

(30) Foreign Application Priority Data
Jul. 14, 2006 (US) 60831007

(51) Int. Cl. A61K 31/52; C07H 19/167; A61P 43/00

(52) U.S. Cl. 514/46; 536/27.62

(57) ABSTRACT

The present application discloses locked nucleoside compounds of the Formula I which act as antagonists of adenosine receptors, in particular the adenosine A3 receptor, and the use of such adenosine A3 receptor compounds in medicine, e.g. for the treatment or alleviation or prophylaxis of selected from the group consisting of pain; inflammatory diseases, arthritis, multiple sclerosis, inflammation, asthma and psoriasis; gastro-intestinal disorders; allergy; disorders associated with mast cell or eosinophil activation and degranulation; cardio-vascular disorders; cutaneous diseases; wound healing; opthalmological disorders; respiratory disorders; kidney diseases; central nervous system disorders; Alzheimer's disease, Creutzfeldt-Jacob disease, Huntington's disease and Parkinson's disease; trauma and seizure; diabetes; osteoporosis; diseases of the immune system; cancers, infections; high blood pressure, locomotor hyperactivity, hypertension and depression; acute hypoxia; neonatal hypoxia, hypoxia and chronic hypoxia; and infertility.

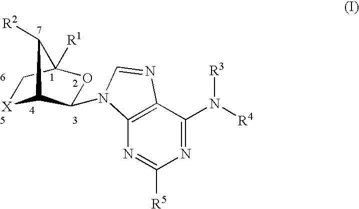

### FIELD OF THE INVENTION

The present invention provides compounds which act as antagonists of adenosine receptors, such as the adenosine A₃ receptor, and the use of the adenosine A₃ receptor compounds in medicine.

### BACKGROUND OF THE INVENTION

The adenosine receptors (AR) are members of the superfamily of G-protein coupled receptors. Four distinct subtypes of ARs have been characterized, the A₁, A_{2A}, A_{2B} and A₃ subtype respectively. The adenosine receptors are widely distributed throughout the body and play a major role in the regulation of many organs. All four receptors are activated by adenosine generated by the degradation of ATP in metabolically active cells. The A₁ and A₃ receptors are coupled to adenylate cyclase activity and activation leads to a decrease in the cAMP level and an increase in the intracellular levels of calcium. Activation of the A_{2A} and A_{2B} receptors on the other hand leads to an increase in cAMP levels. Both the A₁ and A_{2A} receptors play important roles in the central nervous system and cardiovascular system. In the CNS, adenosine inhibits the release of synaptic transmitters which effect is mediated by A₁ receptors. In the heart, the A₁ receptors mediate the negative inotropic, chronotropic and dromotropic effects of adenosine. The adenosine A_{2A} receptors display functional interaction with dopamine receptors in regulating the synaptic transmission. The A_{2B} receptors on endothelial and smooth muscle cells are responsible for adenosine-induced vasodilation. Expression levels for A₃ receptors are rather low compared to other subtypes and they are highly species dependent. A₃ receptors are expressed primarily in the CNS, in the testis and in the immune system, and appear to be involved in the modulation of the mediator release from the mast cells in immediate hypersensitivity reaction.

Adenosine receptors are considered to play a basic role in the different pathologies such as inflammation and neurodegeneration, ischemic brain damage, cardiac ischemia, hypertension, ischemic heart pre-conditioning, asthma and cancer.

Proc. Natl. Acad. Sci. USA, 1992, 89, 7432-7436 discloses that adenosine itself is an agonist of the adenosine A₃ receptor.

WO 95/02604 discloses adenosine analogs modified at N⁶, C-2 and C-5′, in particularly substituted N⁶-benzyladenosine-5′-uronamides, as adenosine A₃ receptor agonists. Therefore it is known that adenosine analogues modified at N⁶, C-2 and C-5′ may be adenosine receptor agonists.

J. Med. Chem., 2003, 46, 353-355, discloses N⁶-alkylated 3′-deoxy-3-amino-adenosine-5′-uronamides as adenosine A₃ receptor agonists. Therefore it is known that adenosine analogues modified with an amino group at C-3′ may be adenosine receptor agonists.

J. Med. Chem., 2006, 49, 2689-2702 discloses N⁶-alkylated-adenosine-5′-uronamides and N⁶-alkylated-adenosines substituted at C-3′ with amino, azido, ureido and aminomethyl groups with diminished agonist activity at the adenosine A₃ receptor.

J. Med. Chem., 2002, 45, 4471-4484 and Bioorg. Med. Chem. Lett., 2006, 16, 596-601, disclose that some adenosine-5′-uronamides can be converted from adenosine A₃ receptor agonists into antagonists by certain modifications.

J. Med. Chem., 2000, 43, 2196-2203 on the other hand discloses that restriction of the ribose ring of adenosine-derived agonists into an N-type conformation using a methanocarba-adenosine scaffold generally increases the agonistic effect.

It is believed that the following compounds are known in the art and have been used in the preparation of therapeutic oligonucleotides:

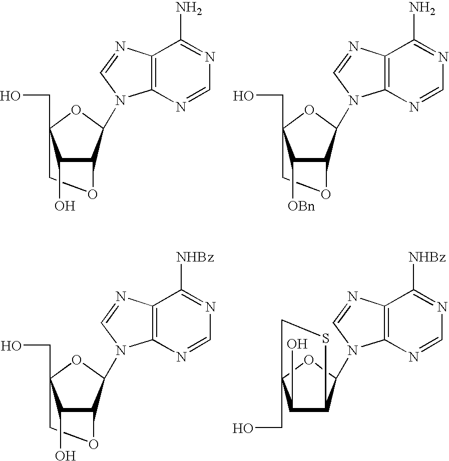

It has not been suggested that they may be used per se in medicine, in particular not as adenosine A₃ receptor antagonists.

### SUMMARY OF THE INVENTION

The present invention provides compounds of the Formula I for use as a medicament

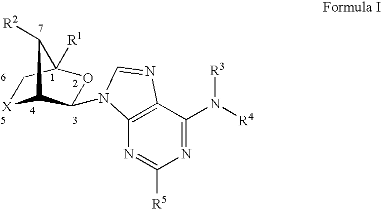

wherein:
X is selected from the group consisting of —O—, —S—, >NH and >NR′, wherein R′ is selected from the group consisting of hydrogen, C₁-C₆ acyl and C₁-C₆ alkyl;
R¹ is selected from the group consisting of R^{a}R^{b}NC(═O)— and HOR^{c}—, wherein R^{a} and R^{b} are independently selected from the group consisting of hydrogen, optionally substituted C₁-C₁₀ alkyl (including C₁-C₁₀ haloalkyl, amino-C₁-C₁₀ alkyl, Boc-amino-C₁-C₁₀ alkyl, and C₃-C₁₀ cycloalkyl), optionally substituted C₁-C₁₀ acyl, formyl, optionally substituted aryl, and optionally substituted arylcarbonyl; or R^{a} and R^{b} may together with the nitrogen atom to which they are attached form an optionally substituted 4- to 8-membered heterocyclic ring; R^{c} is selected from the group consisting of C₁-C₆ alkylene (including C₃-C₆ cycloalkylene), C₁-C₆ haloalkylene, and carbonyl;
R² is selected from the group consisting of hydrogen, hydroxyl, amino, azido, halo, optionally substituted C₁-C₆ alkyl, optionally substituted C₁-C₆ alkoxy, carboxy, nitrilo, nitro, aryl, thiol, and —Y—CO—R^{d}, wherein Y is selected from the group consisting of —O—, >NH and S—, and R^{d} is selected from the group consisting of —NH₂, —OH and C₁-C₆ alkyl;
R³ and R⁴ are independently selected from the group consisting of hydrogen, optionally substituted C₁-C₁₀ alkyl (including phenylethyl (including R- and S-1-phenylethyl), benzyl, C₁-C₁₀ haloalkyl, amino-C₁-C₁₀ alkyl, Boc-amino-C₁-C₁₀ alkyl, and C₃-C₁₀ cycloalkyl), optionally substituted C₁-C₁₀ alkoxycarbonyl, optionally substituted C₁-C₁₀ acyl, formyl, mono- and di(C₁-C₁₀ alkyl)aminocarbonyl, C₁-C₁₀ alkylsulphonyl, C₁-C₁₀ alkylsulphinyl, optionally substituted aryl, optionally substituted arylcarbonyl, optionally substituted heterocyclyl, optionally substituted heterocyclylcarbonyl, optionally substituted heteroaryl, heteroarylcarbonyl; or R³ and R⁴ may together with the nitrogen atom to which they are attached form an optionally substituted 4- to 8-membered heterocyclic ring;
R⁵ is selected from the group consisting of hydrogen, halogen (such as chlorine, iodine or bromine), optionally substituted C₁-C₁₀ alkyl, optionally substituted C₂-C₆ alkenyl, optionally substituted C₂-C₆ alkynyl, hydroxyl, optionally substituted C₁-C₁₀ alkoxy, amino, optionally substituted C₁-C₁₀ alkylamino, mercapto, and optionally substituted C₁-C₁₀ alkylthio;
and wherein the stereocentres 1, 3, 4 and 7 may be present in either orientation.

The present invention further provides various methods of treatment/therapy comprising administering a compound of formula I to a patient, as well as the use of a compound of Formula I in the manufacture of a medicament for the treatment or prophylaxis of an adenosine A₃ receptor related disease.

The present invention further provides a compound of formula I,

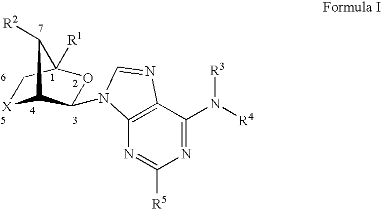

as defined herein, wherein, either, said compound is not selected from one of the following:

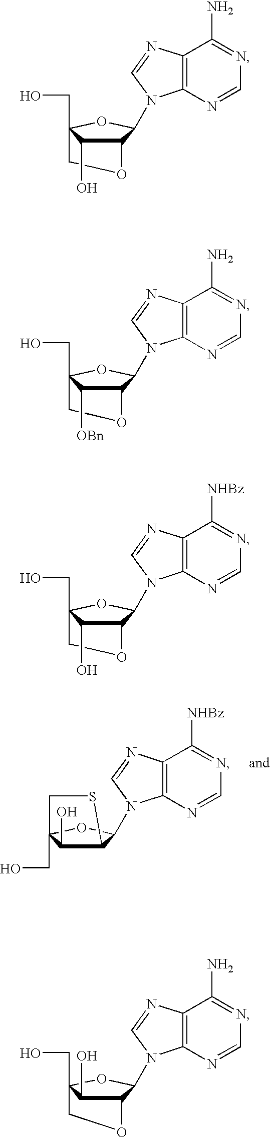

or wherein when R¹ is —CH₂OH and R² is —OH, either (i) R³, R⁴ and R⁵ are not all H, or (ii) X is not selected from the group consisting of —O— and —S—.

### BRIEF DESCRIPTION OF THE FIGURES

FIG. 1 illustrates the synthesis of SPN0171, SPN0159, SPN0186, SPN1078 and SPN169. Method A: i) TEMPO, diacetoxyiodobenzene, CH₃CN/H₂O, 40° C., ii) SOCl₂, EtOH, RT, iii) MeNH₂, MeOH, RT. Method B: MeOH, HCO₂NH₄, Pd(OH)₂/C, reflux. Method C: i) Alkyl bromide or alkyl chloride, DMF, 70° C., ii) NH₄OH, MeOH, 50° C.

FIG. 2 illustrates the synthesis of SPN 0174, SPN 0175, SPN 0176, SPN0196 and SPN0197. Method A: i) TEMPO, diacetoxyiodobenzene, CH₃CN/H₂O, 40° C., ii) SOCl₂, EtOH, RT, iii) MeNH₂, MeOH, RT. Method D: MeSO₂OH, DCM, 0° C.

FIG. 3 illustrates the synthesis of SPN0238, SPN0234 and SPN 0234. Method A: i) TEMPO, diacetoxyiodobenzene, CH₃CN/H₂O, 40° C., ii) SOCl₂, EtOH, RT, iii) MeNH₂, MeOH, RT. Method B: MeOH, HCO₂NH₄, Pd(OH)₂/C, reflux. Method C: i) Alkyl bromide or alkyl chloride, DMF, 70° C., ii) NH₄OH, MeOH, 50° C. Method D: MeSO₂OH, DCM, 0° C.

FIG. 4 illustrates the synthesis of SPN0252, SPN0190, SPN0191, SPN0192 and SPN0195. Method A: i) TEMPO, diacetoxyiodobenzene, CH₃CN/H₂O, 40° C., ii) SOCl₂, EtOH, RT, iii) MeNH₂, MeOH, RT. Method B: MeOH, HCO₂NH₄, Pd(OH)₂/C, reflux. Method C: i) Alkyl bromide or alkyl chloride, DMF, 70° C., ii) NH₄OH, MeOH, 50° C.

FIG. 5 illustrates the synthesis of SPN0240, SPN0241 and SPN241. Method A: i) TEMPO, diacetoxyiodobenzene, CH₃CN/H₂O, 40° C., ii) SOCl₂, EtOH, RT, iii) MeNH₂, MeOH, RT. Method B: MeOH, HCO₂NH₄, Pd(OH)₂/C, reflux. Method C: i) Alkyl bromide or alkyl chloride, DMF, 70° C., ii) NH₄OH, MeOH, 50° C.

FIG. 6 illustrates the synthesis of SPN0200, SPN0201, SPN0170, SPN199 and SPN0243. Method A: i) TEMPO, diacetoxyiodobenzene, CH₃CN/H₂O, 40° C., ii) SOCl₂, EtOH, RT, iii) MeNH₂, MeOH, RT. Method B: MeOH, HCO₂NH₄, Pd(OH)₂/C, reflux. Method C: i) Alkyl bromide or alkyl chloride, DMF, 70° C., ii) NH₄OH, MeOH, 50° C.

FIG. 7 illustrates a potential route for the synthesis of compounds of this invention where X is —S—.

### DESCRIPTION OF THE INVENTION

### Definitions

In the present context, the term “C₁-C₁₀ alkyl” is intended to mean a linear, cyclic or branched hydrocarbon group having 1 to 10 carbon atoms, such as methyl, ethyl, propyl, iso-propyl, cyclopropyl, butyl, iso-butyl, tert-butyl, cyclobutyl, pentyl, cyclopentyl, hexyl, cyclohexyl, etc. The cyclic variants hereof are often referred to as “cycloalkyl”, more particular “C₃-C₁₀ cycloalkyl”. “C₁-C₆ alkyl” (which is often preferred)—of course—refers to shorter variants having 1 to 6 carbon atoms.

Similarly, the term “C₂-C₆ alkenyl” is intended to cover linear, cyclic or branched hydrocarbon groups having 2 to 6 carbon atoms and comprising one unsaturated double bond. Examples of alkenyl groups are vinyl, allyl, butenyl, pentenyl, hexenyl, heptenyl, octenyl, hepta-decaenyl. Preferred examples of alkenyl are vinyl, allyl, butenyl, especially allyl. The term “C₂-C₆ alkynyl” is intended to cover linear, cyclic or branched hydrocarbon groups having 2 to 6 carbon atoms and comprising one unsaturated triple bond.

Moreover, the term “C₁-C₆ alkylene” is intended to mean a linear, cyclic or branched hydrocarbon biradical having 1 to 6 carbon atoms, such as methylene, ethylene, propylene, iso-propylene, cyclopropylene, butylene, iso-butylene, tert-butylene, cyclobutylene, pentylene, cyclopentylene, hexylene, cyclohexylene, etc. The cyclic variants hereof are often referred to as “cycloalkylene”, more particular “C₃-C₆ cycloalkylene”.

The terms “haloalkyl” and “haloalkylene” are intended to mean alkyl and alkylene, respectively, being substituted with one or more halogen atoms, e.g. one, two, three or four halogen atoms, or even halogen atoms corresponding to all hydrogen atoms of the alkylene (perhalogenation).

The term “acyl” means alkylcarbonyl, e.g. “C₁-C₆ acyl” means C₁-C₆ alkyl-carbonyl.

The term “alkoxy” means alkyloxy, e.g. “C₁-C₆ alkoxy” means C₁-C₆ alkyl-oxy.

In the present context, i.e. in connection with the terms “alkyl”, “alkylene”, “alkoxy”, “acyl”, “alkenyl”, and “alkynyl”, the term “optionally substituted” is intended to mean that the group in question may be substituted one or several times, preferably 1-3 times, with group(s) selected from hydroxy (which when bound to an unsaturated carbon atom may be present in the tautomeric keto form), C₁-C₆ alkoxy, C₂-C₆ alkenyloxy, carboxy, oxo (forming a keto or aldehyde functionality), C₁-C₆ alkoxycarbonyl, C₁-C₆ acyl, formyl, aryl, aryloxy, arylamino, arylcarbonyl, aryloxycarbonyl, arylcarbonyloxy, arylaminocarbonyl, arylcarbonylamino, heteroaryl, heteroaryloxy, heteroarylamino, heteroarylcarbonyl, heteroaryloxycarbonyl, heteroarylcarbonyloxy, heteroarylaminocarbonyl, heteroarylcarbonylamino, heterocyclyl, heterocyclyloxy, heterocyclylamino, heterocyclylcarbonyl, heterocyclyloxycarbonyl, heterocyclylcarbonyloxy, heterocyclylaminocarbonyl, heterocyclylcarbonylamino, amino, mono- and di(C₁-C₆ alkyl)amino, carbamoyl, mono- and di(C₁-C₆ alkyl)aminocarbonyl, C₁-C₆ acylamino, cyano, guanidino, carbamido, C₁-C₆ alkyl-sulphonyl-amino, aryl-sulphonyl-amino, heteroaryl-sulphonyl-amino, C₁-C₆ alkanoyloxy, C₁-C₆ alkyl-sulphonyl, C₁-C₆ alkyl-sulphinyl, C₁-C₆ alkylsulphonyloxy, nitro, C₁-C₆ alkylthio, and halogen, where any aryl, heteroaryl and heterocyclyl may be substituted as specifically described below for aryl, heteroaryl and heterocyclyl, and any alkyl, alkoxy, and the like, representing substituents may be substituted with hydroxy, C₁-C₆ alkoxy, amino, mono- and di(C₁-C₆ alkyl)amino, carboxy, C₁-C₆ acylamino, C₁-C₆ alkylaminocarbonyl, or halogen(s).

Typically, the substituents are selected from hydroxy (which when bound to an unsaturated carbon atom may be present in the tautomeric keto form), C₁-C₆ alkoxy, C₂-C₆ alkenyloxy, carboxy, oxo (forming a keto or aldehyde functionality), C₁-C₆ acyl, formyl, aryl, aryloxy, arylamino, arylcarbonyl, heteroaryl, heteroaryloxy, heteroarylamino, heteroarylcarbonyl, heterocyclyl, heterocyclyloxy, heterocyclylamino, heterocyclylcarbonyl, amino, mono- and di(Cⱼ—C₆ alkyl)amino; carbamoyl, mono- and di(C₁-C₆ alkyl)aminocarbonyl, amino-C₁-C₆ alkyl-aminocarbonyl, mono- and di(C₁-C₆ alkyl)amino-C₁-C₆ alkyl-aminocarbonyl, C₁-C₆ acylamino, guanidino, carbamido, C₁-C₆ alkyl-sulphonyl-amino, C₁-C₆ alkyl-sulphonyl, C₁-C₆ alkyl-sulphinyl, C₁-C₆ alkylthio, halogen, where any aryl, heteroaryl and heterocyclyl may be substituted as specifically described below for aryl, heteroaryl and heterocyclyl.

In some embodiments, substituents are selected from hydroxy, C₁-C₆ alkoxy, amino, mono- and di(C₁-C₆ alkyl)amino, carboxy, C₁-C₆ acylamino, C₁-C₆ alkylaminocarbonyl, and halogen.

The term “halogen” includes fluoro, chloro, bromo, and iodo.

The term “Boc” means tert-butoxycarbonyl, i.e. an N-protecting group.

In the present context, the term “aryl” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The term “heteroaryl” is intended to mean a fully or partially aromatic carbocyclic ring or ring system where one or more of the carbon atoms have been replaced with heteroatoms, e.g. nitrogen (═N— or —NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furanyl, thienyl, quinolyl, benzothiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are benzimidazolyl, oxazolyl, isoxazolyl, thiazolyl, isothiazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular benzimidazolyl, pyrrolyl, imidazolyl, pyridinyl, pyrimidinyl, furyl, thienyl, quinolyl, tetrazolyl, and isoquinolyl.

The term “heterocyclyl” is intended to mean a non-aromatic carbocyclic ring or ring system where one or more of the carbon atoms have been replaced with heteroatoms, e.g. nitrogen (═N— or —NH—), sulphur, and/or oxygen atoms. Examples of such heterocyclyl groups (named according to the rings) are imidazolidine, piperazine, hexahydropyridazine, hexa hydropyrimidine, diazepane, diazocane, pyrrolidine, piperidine, azepane, azocane, aziridine, azirine, azetidine, pyrroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are tetrahydrofuran, imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular tetrahydrofuran, imidazolidine, piperazine, hexahydropyridazine, hexahydropyrimidine, diazepane, pyrrolidine, piperidine, azepane, oxazinane (morpholine), and thiazinane.

The expression “4- to 8-membered heterocyclic ring” is intended to means a ring of the type specified above under “heteroaryl” and “heterocyclyl” provided that the ring comprises 4 to 8 ring atoms.

In the present context, i.e. in connection with the terms “aryl”, “heteroaryl”, “heterocyclyl”, “4- to 8-membered heterocyclic ring”, and the like (e.g. “aryloxy”, “heterarylcarbonyl”, etc.), the term “optionally substituted” is intended to mean that the group in question may be substituted one or several times, preferably 1-5 times, in particular 1-3 times, with group(s) selected from hydroxy (which when present in an enol system may be represented in the tautomeric keto form), C₁-C₆ alkyl, C₁-C₆ alkoxy, C₂-C₆ alkenyloxy, oxo (which may be represented in the tautomeric enol form), carboxy, C₁-C₆ alkoxycarbonyl, C₁-C₆ acyl, formyl, aryl, aryloxy, arylamino, aryloxycarbonyl, arylcarbonyl, heteroaryl, heteroarylamino, amino, mono- and di(C₁-C₆ alkyl)amino; carbamoyl, mono- and di(C₁-C₆ alkyl)aminocarbonyl, amino-C₁-C₆ alkyl-aminocarbonyl, mono- and di(C₁-C₆ alkyl)amino-C₁-C₆ alkyl-aminocarbonyl, C₁-C₆ acylamino (e.g. acetamido), cyano, guanidino, carbamido, C₁-C₆ alkanoyloxy, C₁-C₆ alkyl-sulphonyl-amino, aryl-sulphonyl-amino, heteroaryl-sulphonyl-amino, C₁-C₆ alkyl-suphonyl, C₁-C₆ alkyl-sulphinyl, C₁-C₆ alkylsulphonyloxy, nitro, sulphanyl, amino, amino-sulfonyl, mono- and di(C₁-C₆ alkyl)amino-sulfonyl, halogen-C₁-C₄ alkyl, dihalogen-C₁-C₄ alkyl, trihalogen-C₁-C₄ alkyl, halogen, where aryl and heteroaryl representing substituents may be substituted 1-3 times with C₁-C₄ alkyl, C₁-C₄ alkoxy, nitro, cyano, amino or halogen, and any alkyl, alkoxy, and the like, representing substituents may be substituted with hydroxy, C₁-C₆ alkoxy, C₁-C₆ alkenyloxy, amino, mono- and di(C₁-C₆ alkyl)amino, carboxy, C₁-C₆ acylamino, halogen, C₁-C₆ alkylthio, C₁-C₆ alkyl-sulphonyl-amino, or guanidino.

Typically, the substituents are selected from hydroxy, C₁-C₆ alkyl, C₁-C₆ alkoxy, oxo (which may be represented in the tautomeric enol form), carboxy, C₁-C₆ acyl, formyl, amino, mono- and di(C₁-C₆ alkyl)amino; carbamoyl, mono- and di(C₁-C₆ alkyl)aminocarbonyl, amino-C₁-C₆ alkyl-aminocarbonyl, C₁-C₆ acylamino, guanidino, carbamido, C₁-C₆ alkyl-sulphonyl-amino, aryl-sulphonyl-amino, heteroaryl-sulphonyl-amino, C₁-C₆ alkyl-suphonyl, C₁-C₆ alkyl-sulphinyl, C₁-C₆ alkylsulphonyloxy, sulphanyl, amino, amino-sulfonyl, mono- and di(C₁-C₆ alkyl)amino-sulfonyl or halogen, where any alkyl, alkoxy and the like, representing substituents may be substituted with hydroxy, C₁-C₆ alkoxy, C₂-C₆ alkenyloxy, amino, mono- and di(C₁-C₆ alkyl)amino, carboxy, C₁-C₆ acylamino, halogen, C₁-C₆ alkylthio, C₁-C₆ alkyl-sulphonyl-amino, or guanidino. In some embodiments, the substituents are selected from C₁-C₆ alkyl, C₁-C₆ alkoxy, amino, mono- and di(C₁-C₆ alkyl)amino, sulphanyl, carboxy or halogen, where any alkyl, alkoxy and the like, representing substituents may be substituted with hydroxy, C₁-C₆ alkoxy, C₂-C₆ alkenyloxy, amino, mono- and di(C₁-C₆ alkyl)amino, carboxy, C₁-C₆ acylamino, halogen, C₁-C₆ alkylthio, C₁-C₆ alkyl-sulphonyl-amino, or guanidino.

The term “prodrug” used herein is intended to mean a derivative of a compound of the formula (I) which—upon exposure to physiological conditions—will liberate a compound of the formula (I) which then will be able to exhibit the desired biological action. Examples of prodrugs are esters (carboxylic acid ester, phosphate esters, sulphuric acid esters, etc.), acid labile ethers, acetals, ketals, etc.

The term “pharmaceutically acceptable salts” is intended to include acid addition salts and basic salts. Illustrative examples of acid addition salts are pharmaceutically acceptable salts formed with non-toxic acids. Exemplary of such organic salts are those with maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulfonic, and theophylline acetic acids, as well as the 8-halotheophyllines, for example 8-bromotheophylline. Exemplary of such inorganic salts are those with hydrochloric, hydrobromic, sulfuric, sulfamic, phosphoric, and nitric acids. Examples of basic salts are salts where the (remaining) counter ion is selected from alkali metals, such as sodium and potassium, alkaline earth metals, such as calcium, and ammonium ions (⁺N(R)₃R′, where R and R′ independently designates optionally substituted C₁-C₆ alkyl, optionally substituted C₂-C₆ alkenyl, optionally substituted aryl, or optionally substituted heteroaryl). Pharmaceutically acceptable salts and pharmaceutically acceptable carriers are, e.g., those described in Remington's Pharmaceutical Sciences, 17. Ed. Alfonso R. Gennaro (Ed.), Mack Publishing Company, Easton, Pa., U.S.A., 1985 and more recent editions and in Encyclopedia of Pharmaceutical Technology. Thus, the term “an acid addition salt or a basic salt thereof” used herein is intended to comprise such salts. Furthermore, the compounds as well as any intermediates or starting materials may also be present in hydrate form.

### Compounds of the Formula I

X in Formula I is selected from the group consisting of —O—, —S—, >NH and >NR′, wherein R′ is selected from the group consisting of hydrogen, C₁-C₆ acyl (e.g. acetyl and benzyl) and C₁-C₆ alkyl (e.g. methyl and isopropyl). It is further envisaged that when X is >NH or >NR′, the nitrogen atom may be used as a suitable handle for the design of prodrugs.

Compounds of Formula I may exist in a number of alternative stereochemical forms as the stereocenters 1, 3, 4 and 7 of Formula I gives rise to all in all 16 different stereoisomers of the bicyclic structure. Some of the more interesting and currently explored forms are:

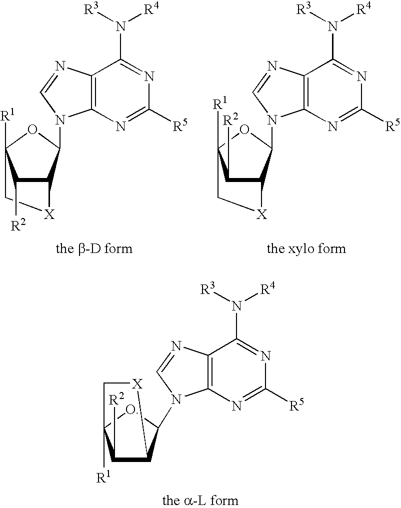

The compounds of the invention may be in the β-D form. In β-D formation, preferably X is either —O— (β-D-oxy) or >NH (β-D-amino).

The compounds of the invention may be in the α-L form. In α-L form, preferably X is —O— (α-L-oxy) or >NH (α-L-amino).

The compounds of the invention may be in the xylo form. In the xylo form, preferably X is —O—.

In a preferable embodiment, R¹ is selected from the group consisting of —CH₂OH, —COON, and R^{a}R^{b}NC(═O)— (e.g. —CONHCH₃).

In one embodiment, R¹ is selected from —CH₂OH, —CO₂H, or R^{a}R^{b}NC(═O)—, wherein R^{a} and R^{b} are independently selected from H and C₁-C₂ alkyl.

In one embodiment, R¹ is —CONHR^{a} or —CONR^{a}R^{b}. When R¹ is —CONHR′, compounds where R^{a} is selected from C₁ alkyl and C₂ alkyl may be particularly effective.

In one interesting embodiment, R¹ is —CONHCH₃.

In one embodiment, R² is —OH. In an alternative embodiment, R² is —N₃. In a further alternative embodiment, R² is —NH₂.

In one embodiment, R³ is H or C₁-C₅ alkyl.

In the same or different embodiments, R⁴ is H or C₁-C₆ alkyl.

In a further embodiment, R⁴ is selected from optionally substituted aryl-C₁-C₄ alkyl, e.g. phenylethyl (such as R- and S-1-phenylethyl), and benzyl, both of which may be substituted with one or more substituents independently selected from the group consisting of C₁-C₆ alkyl, amino, halo, C₁-C₆ haloalkyl, nitro, hydroxyl, acetamido, C₁-C₆ alkoxy, and sulfo. Typically, R³ is H or C₁-C₆ alkyl in this embodiment.

In a more particular embodiment, R⁴ is selected from the group consisting of 3-chlorobenzyl, 3-bromobenzyl, and 3-iodobenzyl. Many compounds where R⁴ is 3-iodobenzyl exhibit very good adenosine A₃ receptor antagonist properties.

In an embodiment, R⁵ may be a halogen atom, such as Cl, Br or I. Compounds where R⁵ is Cl may be particularly effective.

Also included in the class of compounds of the formula I are pharmaceutically acceptable salts and prodrugs thereof.

Compounds of the invention, or for use in the composition of the invention include the following list of examples:

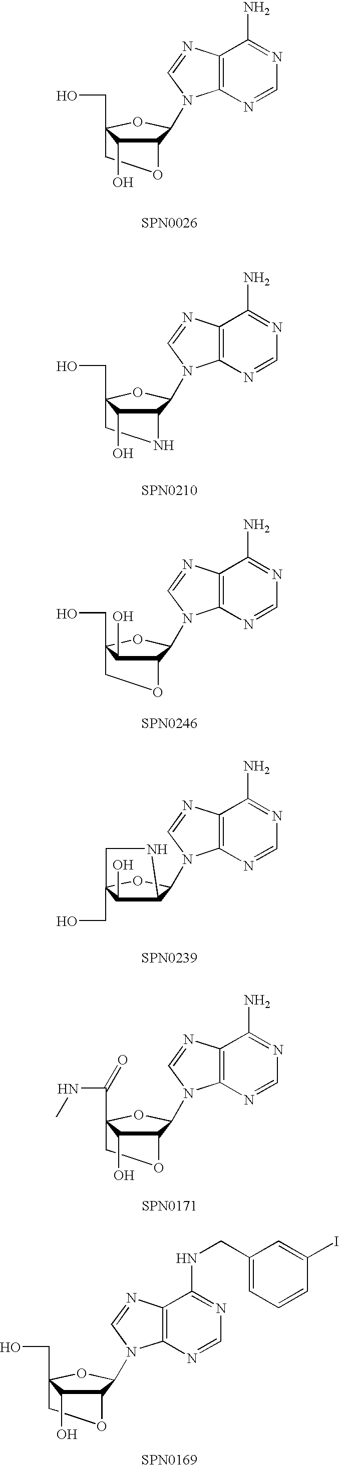

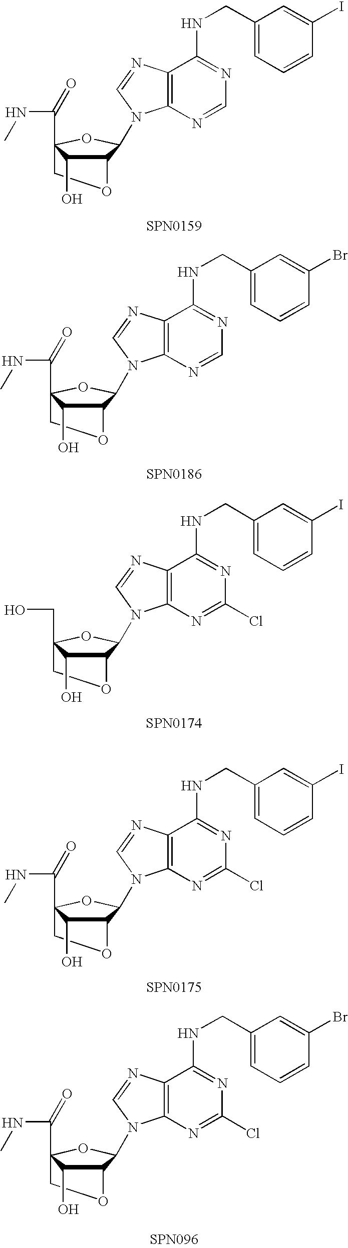

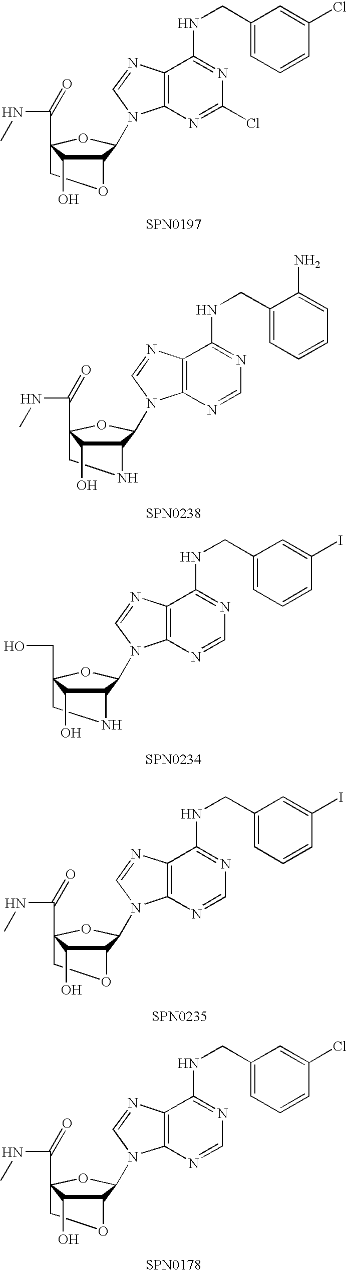

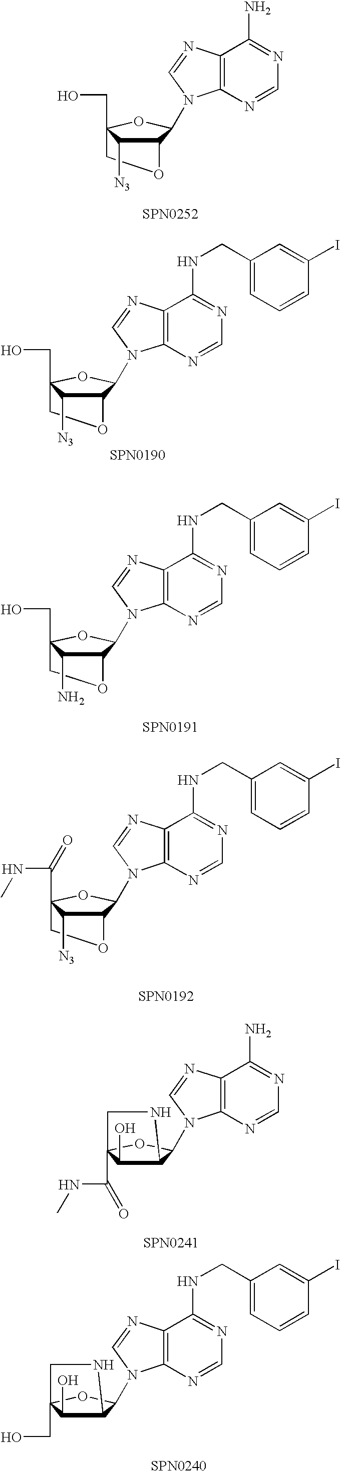

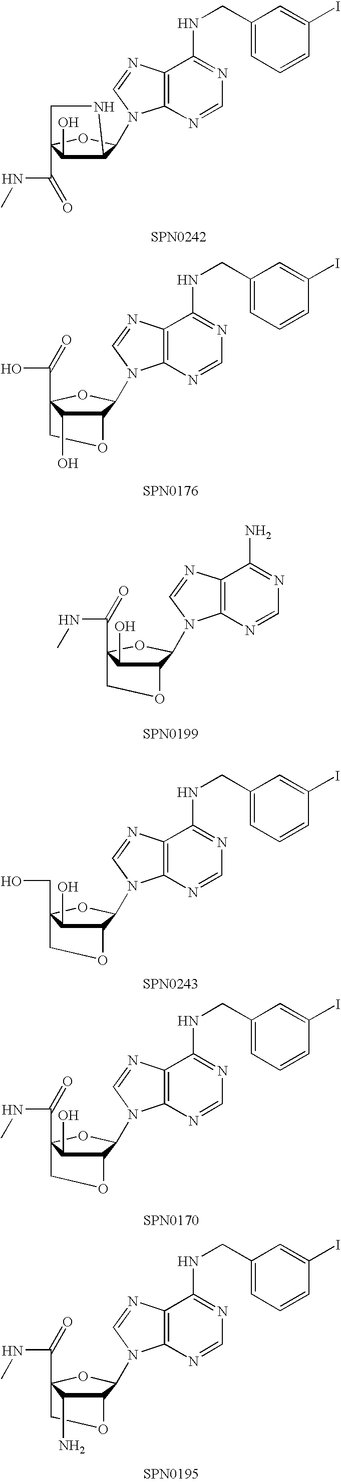

Methods for the synthesis of the above compounds are provided herein (see “Synthesis of Compounds” further below).

Preferred compounds include one or more of the following: SPN0238, SPN0234, SPN0174, SPN0169, SPN0192, SPN0169, SPN0175, SPN0240, SPN0195, SPN0235, and SPN0186.

Preferred compounds include the following β-D-amino compounds: SPN0238, SPN0234, and SPN235.

Preferred compounds include the following β-D-oxy compounds: SPN0174, SPN0175, and SPN0186.

Preferred compounds include the following α-L-amino compound: SPN0240.

Other preferred compounds may include: SPN0192 and SPN0195.

Further compounds of Formula I for use in medicine may include: SPN0210, SPN0159 and SPN0178.

The compounds of Formula I, such as those used within the compositions and or methods of the present invention, may be selected from the group comprising a β-D-oxy, a β-D-amino, a xylo or an α-L-amino locked nucleoside.

In one preferred embodiment, the compound of Formula I is a β-D-amino locked nucleoside.

The compounds of Formula I are found to be adenosine receptor antagonists, in particular adenosine A₃ receptor antagonists. Adenosine A₃ receptor antagonists prevent the decrease in intracellular cAMP caused by activation of the adenosine A₃ receptor by adenosine agonists (for example CI-IB-MECA). Preferably adenosine A₃ receptor antagonists according to the invention have a measured IC₅₀ of 1 μM or less, such as less than 900 nM, such as less than 800 nM, such as less than 700 nM, such as less than 600 nM, such as less than 500 nM, such as less than 400 nM, such as less than 300 nM, such as less than 200 nM, such as less than 100 nM, such as less than 50 nM, such as less than 15 nM. The method for determining the IC₅₀ is provided herein (see Example 41).

### Pharmaceutical Compositions

The compounds may suitable be formulated as a pharmaceutical composition in order to facilitate the proper absorbance of the compound in the relevant region or tissue.

Hence, the invention also provides a pharmaceutical composition comprising compound of Formula I (as defined herein) and a pharmaceutically acceptable carrier.

### Medical Use of Compounds of Formula I

The adenosine receptors antagonised by the compounds and compositions of the invention are preferably mammalian adenosine receptors, most preferably human adenosine receptors.

The compounds and/or compositions of the present invention may be useful for the treatment of adenosine A₃ receptor related diseases.

The expression “adenosine A₃ receptor related disease” is intended to cover any disease or medical conditions which at least in part is causes by or involves a biochemical pathway wherein the adenosine A₃ receptor is included. Such disease and medical disorder may be selected from the group consisting of: acute and chronic pain; inflammatory diseases, inflammatory disorders, arthritis, multiple sclerosis, vascular inflammation, asthma and psoriasis; gastro-intestinal disorders, such as ulcers, inflammatory bowel disease (Crohn's disease) and ulcerative colitis; allergy and allergic responses, eczema, atopic dermatitis and rhinitis; disorders associated with mast cell or eosinophil activation and degranulation, such as asthma, hypesensitivity and allergies; cardio-vascular disorders such as cardiac disease, myocardial infarction, arrhythmias, hypertension, thrombosis, anaemia, arteriosclerosis, angina pectoris, cardiac infarct, and cardiac failure; cutaneous diseases such as urticaria, lupus erythematosus and pruritus; wound healing; opthalmological disorders like glaucoma; respiratory disorders including chronic obstructive pulmonary disease, bronchitis and cystic fibrosis; kidney disease; central nervous system disorders including various forms of epilepsy, stroke, depression and/or sleep apnoea; disorders characterized by impairment of cognition and memory such as Alzheimer's disease, Creutzfeldt-Jacob disease, Huntington's disease and Parkinson's disease; severe neurological disorders related to excitotoxicity, other diseases of the central nervous system, and neurorehabilitation; acute brain or spinal cord injury, trauma and seizure; diabetes; bone diseases such as osteoporosis; diseases of the immune system; cancers, various carcinomas and leukaemia; bacterial and viral infections; high blood pressure, locomotor hyperactivity, hypertension and depression; acute hypoxia; neonatal hypoxia, hypoxia and chronic hypoxia due to arteriovenous malformations and occlusive cerebral artery disease; and infertility.

Hence, the invention provides a method for the treatment of an adenosine A₃ receptor related disease, or for prophylaxis thereof, comprising administering a compound of Formula I, or a pharmaceutical composition comprising a compound of Formula I, to a patient suffering from or at risk of said adenosine A₃ receptor related disease.

In one embodiment, the disease or medical disorder according to the method or use of the invention is selected from the group consisting, an inflammatory disease or disorder, an allergy or allergic response.

In another embodiment, the disease or medical disorder according to the method or use of the invention is selected from the group comprising: Disorders characterized by impairment of cognition and memory such as Alzheimer's disease, Creutzfeldt-Jacob disease, Huntington's disease and/or Parkinson's disease; severe neurological disorders related to excitotoxicity, other diseases of the central nervous system, and neurorehabilitation.

In still another embodiment, the disease or medical disorder according to the method or use of the invention is selected from the group consisting: Acute brain or spinal cord injury, trauma and seizure.

In still another embodiment, the disease or medical disorder according to the method or use of the invention is selected from the group consisting of: cardio-vascular disorders such as cardiac disease, myocardial infarction, arrhythmias, hypertension, thrombosis, anaemia, arteriosclerosis, angina pectoris, cardiac infarct, and cardiac failure.

In still another embodiment, the disease or medical disorder according to the method or use of the invention is selected from the group consisting of: High blood pressure, locomotor hyperactivity, hypertension and depression.

In still another embodiment, the disease or medical disorder according to the method or use of the invention is cancer.

Cancers which may be treated using the compounds/compositions of the invention, and using the methods of the invention include cancers of the lung, breast, colon, prostate, pancreas, lung, liver, thyroid, kidney, brain, testes, stomach, intestine, bowel, spinal cord, sinuses, bladder, urinary tract or ovaries cancer.

The cancer may be in the form of a solid tumor.

The cancer may be a carcinoma, such as carcinoma selected from the group consisting of malignant melanoma, basal cell carcinoma, ovarian carcinoma, breast carcinoma, non-small cell lung cancer, renal cell carcinoma, bladder carcinoma, recurrent superficial bladder cancer, stomach carcinoma, prostatic carcinoma, pancreatic carcinoma, lung carcinoma, cervical carcinoma, cervical dysplasia, laryngeal papillomatosis, colon carcinoma, colorectal carcinoma and carcinoid tumors.

The cancer may be a sarcoma, such as a sarcoma selected from the group consisting of osteosarcoma, Ewing's sarcoma, chondrosarcoma, malignant fibrous histiocytoma, fibrosarcoma and Kaposi's sarcoma.

The cancer may be a glioma.

The method(s) of treatment may be for the treatment of a mammal, such as preferably a human (the patient).

The compounds and/or compositions of the present invention may be combined with chemotherapeutic treatments, for example for the treatment of cancer. The term “combined” is used in this context to mean functionally associated, i.e. it may include combinations of the compound and/or composition of the invention with a chemotherapeutic agent, wherein the chemotherapeutic agent is delivered to the patient in need of treatment either prior to, during and/or subsequent to the delivery of the composition and/or compound of the invention.

In one embodiment the invention provides for chemotherapeutic compositions comprising the compounds of the invention and at least one chemotherapeutic treatment. The chemotherapeutic treatment may be selected from one or more of the following: taxanes, such as paclitaxel (Taxol™) or docetaxel; vinca alkaloids, such as vincrisitine; camptothecin, or a chemotherapeutic antibiotic; preferably Taxanes such as paclitaxel (Taxol™) or docetaxel.

In one embodiment, the compound of Formula I, or the pharmaceutical composition comprising the compound of Formula I, is administered during the administration of chemotherapy treatment.

As described in WO2004/000237, chemotherapeutic treatments can result in severe side effects. This is particularly common in the treatment of cancers.

The invention provides for a method of treatment involving chemotherapy, where in said method comprised administering a compound or composition according to the invention prior to, during and/or subsequent to the administration of one or more chemotherapeutic agents. Such methods may reduce the side effects caused by the chemotherapy treatment and/or allow a greater dose of the chemotherapeutic agent(s) to be used.

Hence, the invention provides a method of reducing or alleviating the detrimental symptoms associated with chemotherapy comprising: administering a compound of Formula I, or a pharmaceutical composition comprising a compound of Formula I, to a patient suffering from or at risk of a disease treatable by chemotherapy, either prior to, during or subsequent to the administration of a chemotherapeutic treatment, so as to reduce or alleviate the detrimental symptoms associated with said chemotherapy treatment.

The invention further provides a method of enhancing a patients tolerance to a chemotherapeutic agent, comprising administering a compound of Formula I, or a pharmaceutical composition comprising a compound of Formula I, to a patient suffering from or at risk of a disease treatable by chemotherapy, either prior to, during or subsequent to the administration of chemotherapy treatment.

The compounds and compositions of the invention may be administered prior to, during and/or subsequent to the administration of one or more chemotherapeutic agents for the treatment for a neoplastic disease.

The invention provides for a method of treatment of a neoplastic disease comprising administering the compound and/or composition according to the invention, such as the chemotherapeutic composition according to the invention, to a patient suffering from a neoplastic disease.

The compounds and compositions of the invention may be administered prior to, during and/or subsequent to the administration of one or more chemotherapeutic agents for the treatment for cancer.

The invention provides for a method of treatment of cancer comprising administering a therapeutically effective amount of the compound and/or composition according to the invention to a patient suffering from cancer.

The compounds and compositions of the invention may be used for the inhibition of eosinophil activation and/or degranulation and thereby prevent conditions such as asthma, hypersensitivity and allergies.

Hence, the invention further provides a method for the inhibition of eosinophil and/or mast cell activation and/or degranulation comprising in a mammal suffering from a disorder or a disease associated with said activation and/or degranulation of said eosinophil and/or mast cells, said method comprising administering a therapeutically effective amount of a compound of Formula I, or a pharmaceutical composition comprising a compound of Formula I, to a mammal so as to inhibit said activation and/or degranulation of said eosinophil and/or mast cell.

In one embodiment, the eosinophil and/or mast cell activation and/or degranulation is associated with a disease selected from one or more of the following: inflammatory diseases, inflammatory disorders, arthritis, multiple sclerosis, vascular inflammation, asthma and psoriasis; gastro-intestinal disorders such as ulcers, inflammatory bowel disease (Crohn's disease) and ulcerative colitis; allergies and allergic responses such as eczema, atopic dermatitis and/or rhinitis.

The invention provides a method for the treatment of disorders associated with eosinophil activation and/or degranulation, such as asthma, hypesensitivity and allergies.

The administration of the compound or composition of the invention may be acute or chronic.

The term “acute” as used herein refers to a period of treatment that last less than 3 months. The term acute includes single dose treatments, and treatments of repeated dosages within a period of less than 3 month which results in substantial alleviation of symptoms and/or cure from the disease or disorder. For acute treatment the prescription period is less than three months.

The term “chronic” as used here refers to a period of treatment (the prescription period) that last for 3 months or more.

The compounds and/or composition of the invention are particularly useful for in vivo applications. For example, A₃ adenosine receptor antagonists can be used in the treatment of any disease, state or condition involving the release of cAMP or the release of inositol-1,4,5-triphosphate, diacylglycerol, and free radicals and subsequent arachidonic acid cascades. Thus, high blood pressure, locomotor hyperactivity, hypertension, acute hypoxia, depression, and infertility can be treated in accordance with the present inventive method.

The present invention provides for a method for the treatment of one or more of the following disorders: high blood pressure; locomotor hyperactivity; hypertension; acute hypoxia; depression; and/or infertility.

More generally, the invention provides a method of deactivating an adenosine receptor in a mammal suffering from an adenosine A₃ receptors related disease, which method comprises administering a therapeutically effective amount of a compound of Formula I, or a pharmaceutical composition comprising a compound of Formula I, to a mammal so as to deactivate the said adenosine receptor.

From a more practical point of view, the invention further provides the use of a compound of Formula I in the manufacture of a medicament for the treatment or prophylaxis of an adenosine A₃ receptors related disease, or for reducing or alleviating the detrimental symptoms associated with chemotherapy.

### Synthesis of Compounds

The following general methods were used:

### General Method A—Conversion of a 5′-OH Nucleoside 5′-Uronamide:

The general method A is useful for the oxidation of the 5′-C position of the compounds of general formula I wherein R¹ is —CH₂OH into a 4′-carboxylic acid (R¹=—CO₂H) and the subsequent conversion into a suitably substituted 4′-uronamide (R¹=R^{a}R^{b}NC(═O)—). This conversion may be conducted even in the presence of secondary and tertiary alcohols, primary or secondary amines in the molecule.

In one variant the starting material is dissolved in polar solvent, e.g. acetonitrile, acetone, water, acetic acid, or mixtures of the same solvents, in particular a mixture of acetonitrile and water in a ratio in the range of 5:1 (v/v) to 1:5 (v/v), e.g. 1:1 (v/v), typically in an amount of 1-100 mL/g, e.g. 10-20 mL/g. To this solution is added 1-10 equivalents of diacetoxyiodobenzene, preferably 2-3 equivalents, and 0.05-1 equivalents of 2,2,6,6-tetramethylpiperidine-1-oxyl (TEMPO), preferably 0.2-0.5 equivalents. The resulting mixture is then stirred at a temperature between 15° C. and 80° C., preferably between 15° C. and 50° C., until full conversion of the starting material to the corresponding carboxylic acid is observed by an analytical method, e.g. LC-MS or TLC, typically after 1-16 hours. In certain cases the product has precipitated from the reaction mixture and can be isolated by filtration followed by washing with an apolar solvent, e.g. diethylether, tert-butyl methyl ether, hexane or heptane, preferably diethylether, and subsequently dried under vacuum. In other cases the solvent is removed under reduced pressure and the residue is triturated with an apolar solvent, e.g. diethylether, tert-butyl methyl ether, hexane or heptane, preferably diethylether, to give the product as a solid or semi-solid material. This product is redissolved in a lower alkyl alcohol solvent, e.g. methanol, ethanol or isopropyl alcohol, preferably ethanol, typically in an amount of 1-100 mL/g, e.g. 10-20 mL/g, and then added 1-20 equivalents of thionyl chloride (SOCl₂), preferably 5-10 equivalents. During addition of SOCl₂ the reaction mixture will preferably be cooled using an ice-water bath to maintain the temperature below 30° C., preferably below 10° C. The reaction mixture is stirred at the initial temperature, or is allowed to reach room temperature, and stirring continued until full conversion of the carboxylic acid to the corresponding alkyl ester, the alkyl depending on the alcohol used as solvent, is observed by an analytical method, e.g. LC-MS or TLC, typically after 3-16 hours. The solvent is removed under reduced pressure and the residue is coevaporated with a protic solvent, e.g. methanol, ethanol or isopropyl alcohol, preferably ethanol. The resulting residue is redissolved in a solution of a primary or secondary amine, e.g. methylamine or ethylamine, in a protic or aprotic solvent, e.g. methanol, ethanol, THF or dioxane, preferably methanol, resulting in a 1-20 equivalent excess of the amine. The solution is stirred at a temperature between 15° C. and 80° C., preferably between 15° C. and 50° C. until full conversion of the ester to the corresponding substituted amide is observed by an analytical method, e.g. LC-MS or TLC, typically after 3-16 hours. The solvent is removed under reduced pressure to give the crude target product, usually as a brown oil or semi-solid.

The following variant (Method A) is used in the examples for the conversion of a 5′-OH nucleoside to a 4′-N-methyl uronamide: The starting material is dissolved in CH₃CN/H₂O (1:1, 10-20 mL/g) and added 3 eq. of diacetoxyiodobenzene and 0.3 eq. of 2,2,6,6-tetramethylpiperidine-1-oxyl (TEMPO) and the resulting solution is stirred at 40° C. until full conversion of starting material is observed by LC-MS, typically 2-16 h. If the product has precipitated from the reaction mixture it is filtered off and washed with Et₂O and dried to give the product, usually as white crystals. In other cases the solvents are removed in vacuo and the resulting oil is triturated with Et₂O and dried to give the product as a solid or semi-solid material. This material is redissolved in EtOH (10-20 mL/g), cooled to 0° C. and then added 10 eq. of SOCl₂. The mixture is stirred at room temperature for 16 h. The solvent is removed in vacuo and the residue co-evaporated with EtOH and then dissolved in a 1M solution of methylamine in MeOH. The resulting mixture is stirred until full conversion to product is observed by LC-MS, typically 2-16 h. The solvent is removed in vacuo to give the crude product, usually as a dark solid material.

### General Method B—Debenzylation Using a Hydrogebn Donor and a Palladium Catalyst:

The general method B is useful for debenzylation of the 3′-O position of the compounds of the general formula I using a hydrogen donor and a palladium catalyst.

In one variant the starting material is dissolved in a protic solvent like methanol, ethanol, water, acetic acid, or mixtures of the same solvents, e.g. methanol, typically in an amount of 1-100 mL/g, e.g. 10-20 mL/g. To this solution is added 1-20 equivalents, preferably 5-10 equivalents, of a hydrogen donor, e.g. ammonium formiate, and a palladium catalyst, e.g. Pd on carbon or Pd(OH)₂ on carbon, typically 0.05 to 0.3 equivalents. The resulting mixture is stirred at a temperature between room temperature and 100° C., preferably at the reflux temperature of the chosen solvent. Stirring is continued until full conversion of the starting material is observed by an analytical method, e.g. LC-MS or TLC, typically after 1-16 hours. The mixture is allowed to reach room temperature and the catalyst is filtered off, either as such, or using a suitable filter agent, e.g. Celite. The filtrate is then concentrated under reduced pressure to give the target product.

The following variant (Method B) is used in the examples for debenzylation using ammonium formate and palladium catalyst: The starting material is dissolved in MeOH (10-20 mL/g) and added 10 eq. of ammonium formate and 0.1 eq. of Pd(OH)₂ (20% on carbon) and the resulting mixture is refluxed until full conversion of starting material is observed by LC-MS, typically 1-16 h. The reaction mixture is allowed to reach room temperature, filtered through a pad of Celite, and evaporated in vacuo to give the product, usually as a white solid material.

### General Method C—Alkylation of N⁶ of an Adenosine Analog:

The general method C is useful for the alkylation of N⁶ of the compounds of general formula I, wherein R³ and R⁴ are both hydrogen, using an alkyl halide and subsequent Dimroth rearrangement under basic conditions.

In one variant the starting material is dissolved in an anhydrous, polar, aprotic solvent, e.g. DMF or DMSO, typically in an amount of 1-100 mL/g, e.g. 10-20 mL/g. To this solution is added 1-10 equivalents, preferably 2-3 equivalents, of an appropriate alkyl halide, e.g. alkyl bromide or alkyl chloride. The resulting mixture is stirred at a temperature between 15° C. and 120° C., preferably between 50° C. and 80° C., until full conversion of the starting material to the corresponding N-1-alkylated intermediate is observed by an analytical method, e.g. LC-MS or TLC, typically after 8-48 hours. If necessary, additional alkyl halide is added during the reaction time. The solvent is removed under reduced pressure and the resulting oil is triturated with a solvent like acetonitrile, acetone, diethylether, tert-butyl methyl ether, hexane or heptane, or mixtures of the same solvents, in particular a mixture of acetone and diethylether in a ratio in the range of 5:1 (v/v) to 1:5 (v/v), e.g. 1:1 (v/v). The residue is dissolved in a polar solvent like ethanol, methanol, acetone or water, e.g. methanol, typically in an amount of 1-100 mL/g, e.g. 10-20 mL/g, and an equal volume of an aqueous basic solution, e.g. concentrated NH₄OH, 5M NaOH or 5M LiOH, in particular concentrated NH₄OH. The resulting mixture is then stirred at a temperature between 15° C. and 80° C., preferably between 40° C. and 50° C., until full conversion of the starting material to the corresponding N6-alkyl product is observed by an analytical method, e.g. LC-MS or TLC, typically after 2-16 hours. The solvent is reduced to ½ volume under reduced pressure and then extracted 1 to 3 times with a non-polar solvent like DCM, diethyl ether, ethyl acetate, or toluene, e.g. DCM. The combined organic phase was removed under reduced pressure to give the crude product.

The following variant (Method C) is used in the examples for the alkylation of N⁶ of an adenosine analog: The starting material is dissolved in anhydrous DMF (10-20 mL/g) and added 3 eq. of the appropriate alkyl bromide or alkyl chloride. The resulting mixture is stirred at 70° C. until full conversion of starting material is observed by LC-MS, typically 16-48 h. If necessary, additional alkylating agent is added during the reaction time. The solvent is removed in vacuo and the resulting oil is triturated with acetone/Et₂O (1:1). The residue is dissolved in MeOH and sat. aq. NH₄OH (3:1, 10-20 mL/g) and stirred at 50° C. for 16 h. The solvent was reduced to ½ volume and then extracted twice with DCM. The combined organic phase was evaporated in vacuo to give the crude product.

### General Method D—Debenzylation Using an Alkylsulfonic Acid:

The general method D is useful for debenzylation at the 3′-O position of the compounds of the general formula I.

In one variant, the starting material is dissolved in a anhydrous aprotic solvent, e.g. anhydrous dichloromethane, dichloroethane, diethyl ether or toluene, in particular dichloromethane, typically in an amount of 1-100 mL/g, e.g. 10-20 mL/g. The solution is kept at a temperature of in the range of from −20 to 50° C., e.g. from −5 to 20° C., or conveniently cooled on an ice bath to about 0° C. A strong acid, e.g. methanesulfonic acid (MsOH), trifluoromethanesulfonic acid, hydrochloric acid, or hydrobromic acid, preferably MsOH, is then added in an amount corresponding to 5-50% of the acid in the solvent, e.g. about a 30% solution in the solvent when MsOH is used. The solution is stirred at the initial temperature, or is allowed to adapt to room temperature. Full conversion of the starting material can be observed by LC-MS, typically after 6-16 h. The reaction mixture is preferably cooled to 0° C. and is neutralized by the drop-wise addition of a tertiary, organic amine (e.g. triethylamine), typically in an amount equivalent to the amount of acid used. The resulting solution is diluted to a volume in the range of 2 to 4 times, e.g. 3 times, the starting volume with an aprotic solvent (e.g. DCM) and is then washed twice with water. The organic phase is evaporated in vacuo to give the crude product as a thick oil or a solid material.

The following variant (Method D) is used in the examples for debenzylation using methanesulfonic acid: The starting material is dissolved in anhydrous DCM (10-20 mL/g), cooled to 0° C. and added methanesulfonic acid to give a 30% solution of MsOH in DCM. The solution is stirred at room temperature until full conversion of starting material is observed by LC-MS, typically 6-16 h. The reaction mixture is again cooled to 0° C. and neutralized by the drop-wise addition of triethylamine. The resulting solution is diluted with DCM and then washed twice with water. The organic phase is evaporated in vacuo to give the crude product as a thick oil or a solid material.

### Method E—Purification by Dry Column Vacuum Chromatography (DCVC):

Crude product is absorbed onto Celite and purified by Dry Column Vacuum Chromatography (DCVC) as described in Synthesis, 2001, 2431-2434 using a suitable eluent gradient.

### Method F—Purification by Medium Pressure Reverse Phase Liquid Chromatography:

Crude product is dissolved in a minimum amount of a mixture of CH₃CN and H₂O (1:1) and applied onto a prepacked column of C18 material. The column was eluted using a gradient of 0-100% solvent A in B, where solvent A is 0.1% NH₄OH in H₂O and solvent B is 20% of A in CH₃CN. The fractions containing product are combined and freeze dried to give the pure product.

### Starting Materials

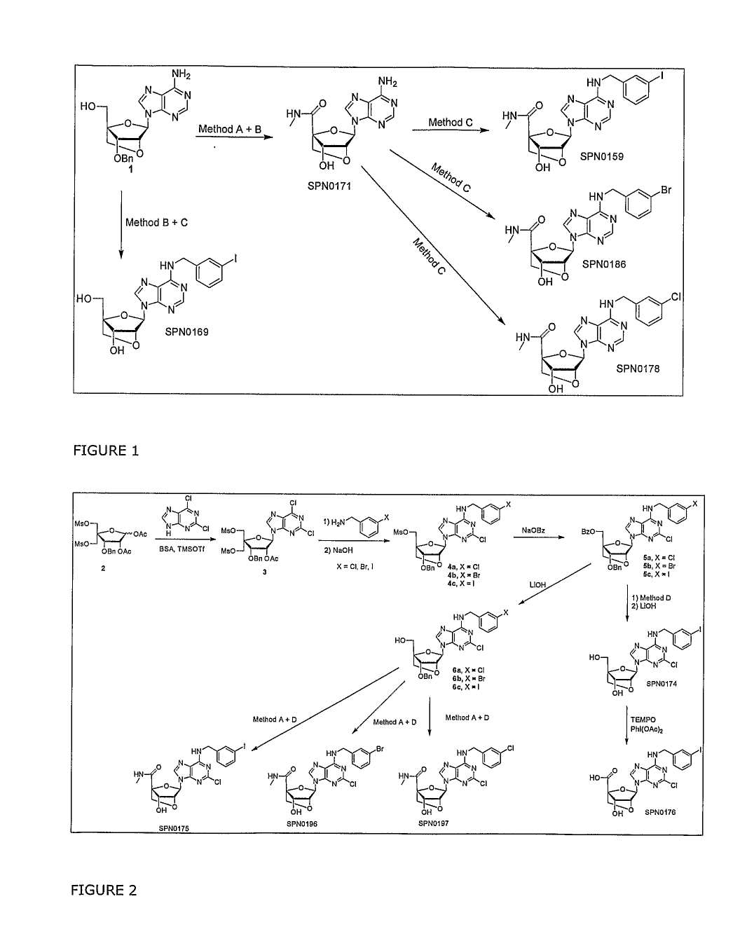

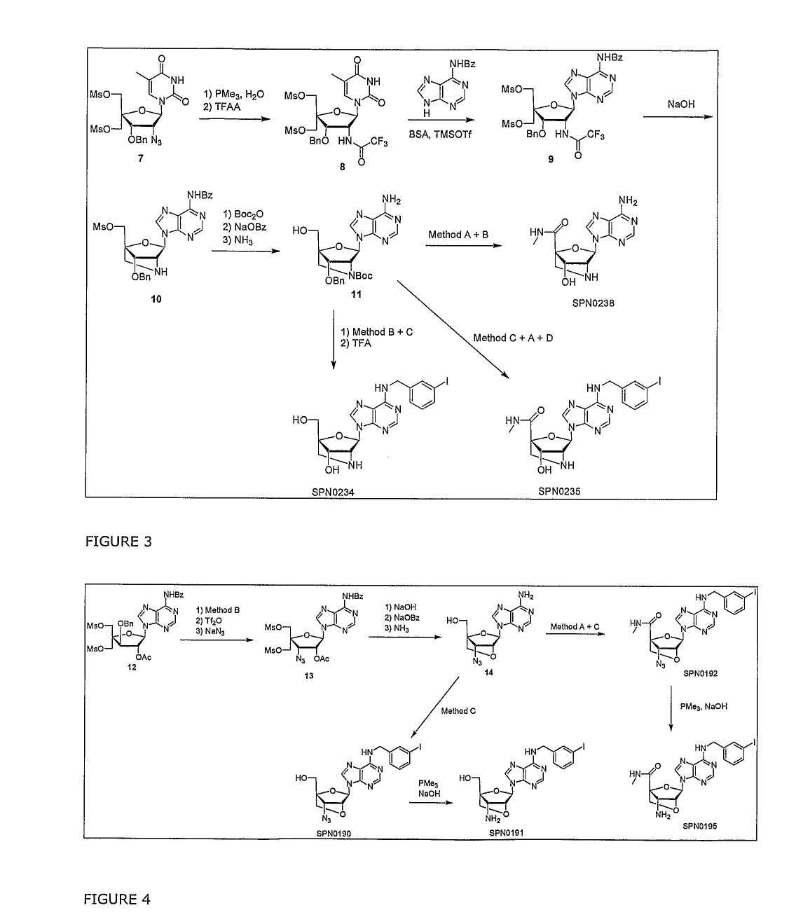

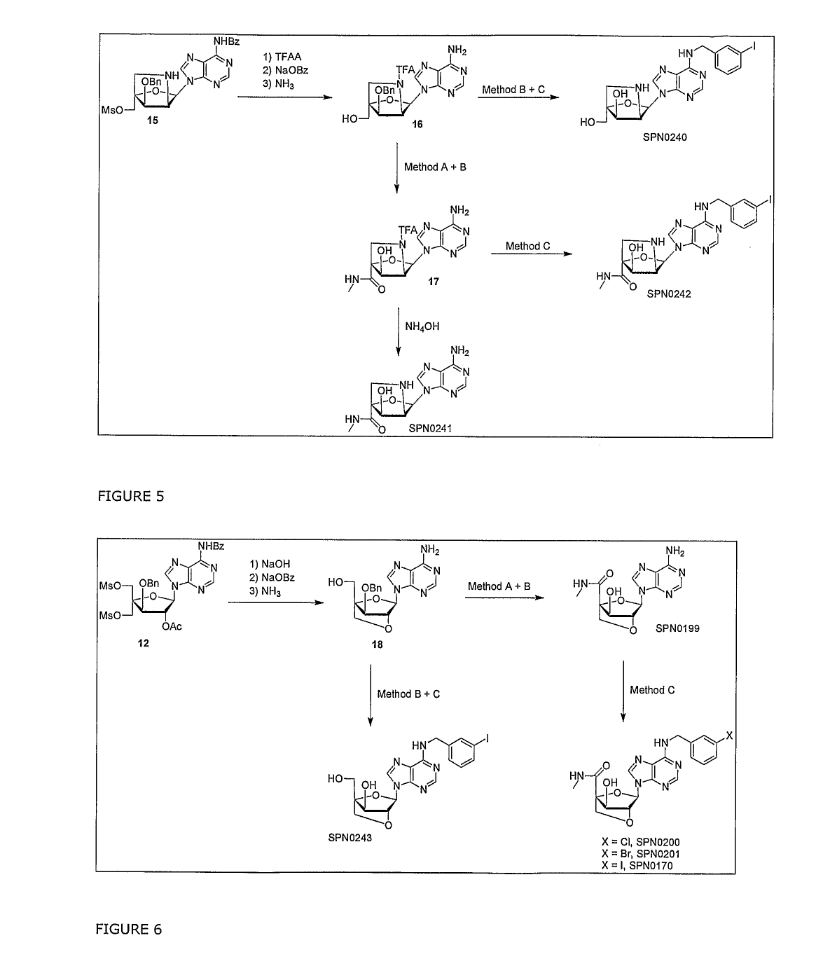

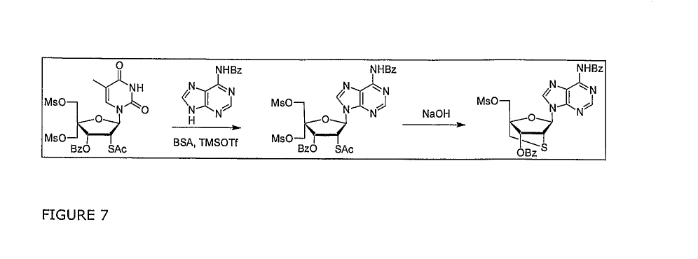

The compounds of the present invention may be prepared from suitable starting material as exemplified in FIG. 1-7. The preparation of these starting materials has been described in detail in the literature. The preparation of compound 1 and 2 is described in detail in J. Org. Chem., 2001, 66, 8504-8512 whereas the preparation of compound 7 is described in detail in Org. Biomol. Chem., 2003, 655-663. Compound 12 can be prepared as described in J. Am. Chem. Soc., 2002, 124, 2164-2176 and compound 15 can be prepared as described in patent application WO03095476. The transformations exemplified in FIG. 1-7 leading to examples of compounds of this invention are known in the art of adenosine derivatives and in the art of Locked Nucleic Acid (LNA)/locked nucleotide chemistry. For example, detailed descriptions on methods of preparation of LNA monomers and manipulations of suitable protecting groups in such compounds can be found in J. Org. Chem., 2001, 66, 8504-8512, Org. Biomol. Chem., 2003, 655-663, J. Am. Chem. Soc., 2002, 124, 2164-2176, and WO/2003095476. For the introduction of modifications at 4′-C, N6 and 2-C of adenosine derivatives thorough descriptions can be found in J. Med. Chem., 2005, 48, 1550-1562 and J. Med. Chem., 1994, 37, 636-646.

### EXPERIMENTALS

### Example 1

### (1S,3R,4R,7S)-7-Hydroxy-1-hydroxymethyl-3-(N6-(3-iodobenzyl)-adenin-9-yl)-2,5-dioxabicyclo[2:2:1]heptane (SPN0169)

(1S,3R,4R,7S)-7-Benzyloxy-1-hydroxymethyl-3-(adenine-9-yl)-2,5-dioxabicyclo[2:2:1]heptane (1, J. Org. Chem., 2001, 66, 8504-8512) was subjected to general method B followed by general method C to give the crude product. Purification by general method E (0-20% MeOH in EtOAc) gave 450 mg of the target compound as a white solid. ¹H-NMR (DMSO-d₆, 300 MHz) δ 8.48 (1H, br s, CH₂NH), 8.27 (1H, s, H-2), 8.22 (1H, s, H-8), 7.72 (1H, br s, 3-I-Ph), 7.59 (1H, d, J=7.9 Hz, 3-I-Ph), 7.37 (1H, d, J=7.7, Hz, 3-I-Ph), 7.10 (1H, t, J=7.8 Hz, 3-I-Ph), 5.92 (1H, s, H-1′), 5.70 (1H, br s, OH), 5.06 (1H, br s, OH), 4.67 (2H, br s, 3-I-PhCH₂), 4.43 (1H, s, H-2′ or H-3′), 4.26 (1H, s, H-2′ or H-3′), 3.92 (1H, d, J=8.2 Hz, Hₐ-1″), 3.79-3.76 (3H, m, H-5′, H_{b}-1″). FAB HR-MS: Theoretical Mass (C18H18IN5O4): 496.04817 (M+H), Measured Mass: 496.047325

### Example 2

### (1R,3R,4R,7S)-(7-hydroxy-3-(adenin-9-yl)-2,5-dioxabicyclo[2:2:1]hept-1-yl)-N-methylcarboxamide (SPN0171)

(1S,3R,4R,7S)-7-Benzyloxy-1-hydroxymethyl-3-(adenine-9-yl)-2,5-dioxabicyclo[2:2:1]heptane (1, J. Org. Chem., 2001, 66, 8504-8512) was subjected to general method A followed by general method B to give the crude product. Purification by general method F gave 75 mg of the target compound as a white solid. ¹H-NMR (DMSO-d₆, 300 MHz) δ 8.36 (1H, s, H-2), 8.30 (1H, q, J=4.7 Hz, NHMe), 8.18 (1H, s, H-8), 7.27 (2H, s, NH₂), 6.01 (1H, br s, OH), 6.00 (1H, s, H-1′), 4.60 (1H, s, H-2′), 4.57 (1H, s, H-3′), 4.35 (1H, d, J=8.0 Hz, Hₐ-1″), 3.91 (1H, d, J=8.0 Hz, H_{b}-1″), 2.68 (3H, d, J=4.7 Hz, CH₃). CI HR-MS: Theoretical Mass (C12H14N6O4): 307.11547 (M+H), Measured Mass: 307.115598.

### Example 3

### (1R,3R,4R,7S)-(7-hydroxy-3-(N6-(3-iodobenzyl)-adenin-9-yl)-2,5-dioxabicyclo-[2:2:1]hept-1-yl)-N-methylcarboxamide (SPN0159)

SPN0171 was subjected to general method C using 3-iodobenzyl bromide to give the crude product. Purification by general method F gave 40 mg of the target compound as a white solid. ¹H-NMR (CDCl₃, 300 MHz) δ 8.40 (1H, s, H-2), 7.84 (1H, s, H-8), 7.72 (1H, s, CH₂NH), 7.62 (1H, d, J=7.9 Hz, 3-I-Ph), 7.39 (1H, d, J=7.3 Hz, 3-I-Ph), 7.26 (1H, s, 3-I-Ph), 7.06 (1H, t, J=7.8 Hz, 3-I-Ph), 6.89 (1H, br s, NHMe), 6.27 (1H, br s, OH), 6.10 (1H, s, H-1′), 4.83 (2H, br s, 3-I-PhCH₂), 4.80 (1H, s, H-2′ or H-3′), 4.75 (1H, s, H-2′ or H-3′), 4.54 (1H, d, J=8.4 Hz, Hₐ-1″), 4.10 (1H, d, J=8.4 Hz, H_{b}-1″), 2.90 (3H, d, J=5.0 Hz, CH₃). FAB HR-MS: Theoretical Mass (C19H19IN6O4): 523.05907 (M+H), Measured Mass: 523.05852.

### Example 4

### (1R,3R,4R,7S)-(7-hydroxy-3-(N6-(3-chlorobenzyl)-adenin-9-yl)-2,5-dioxabicyclo-[2:2:1]hept-1-yl)-N-methylcarboxamide (SPN0178)

SPN0171 was subjected to general method C using 3-chlorobenzyl bromide to give the crude product. Purification by general method E (1-5% MeOH in DCM) gave 29 mg of the target compound as a white solid. ¹H-NMR (DMSO-d₆, 300 MHz) δ 8.54 (1H, br s, CH₂NH), 8.40 (1H, s, H-2), 8.25 (1H, s, H-8), 8.25 (1H, br s, NHMe), 7.38-7.25 (4H, m, 3-Cl-Ph), 6.05 (1H, br s, OH), 6.01 (1H, s, H-1′), 4.70 (2H, br s, 3-Cl-PhCH₂), 4.62 (1H, s, H-2′), 4.58 (1H, br s, H-3′), 4.36 (1H, d, J=8.0 Hz, Hₐ-1″), 3.91 (1H, d, J=8.0 Hz, H_{b}-1″), 2.68 (3H, br s, CH₃). FAB HR-MS: Theoretical Mass (C19H19ClN6O4): 431.12345 (M+H), Measured Mass: 431.123679.

### Example 5

### (1R,3R,4R,7S)-(7-hydroxy-3-(N6-(3-bromobenzyl)-adenin-9-yl)-2,5-dioxabicyclo[2:2:1]hept-1-yl)-N-methylcarboxamide (SPN0186)

SPN0171 was subjected to general method C using 3-bromobenzyl bromide to give the crude product. Purification by general method E (0-5% MeOH in DCM) gave 78 mg of the target compound as a white solid. ¹H-NMR (DMSO-d₆, 300 MHz) δ 8.54 (1H, br s, CH₂NH), 8.41 (1H, s, H-2), 8.26 (1H, s, H-8), 8.25 (1H, br s, NHMe), 7.53 (1H, br s, 3-Br-Ph), 7.41 (1H, ddd, J=7.8, 1.9 and 1.3 Hz, 3-Br-Ph), 7.35 (1H, dt, J=7.8 and 1.3 Hz, 3-Br-Ph), 7.26 (1H, t, J=7.8 Hz, 3-Br-Ph), 6.01 (1H, s, H-1′), 5.95 (1H, br d, J=4.2 Hz, OH), 4.69 (2H, br s, 3-Br-PhCH₂), 4.62 (1H, s, H-2′), 4.59 (1H, br s, H-3′), 4.34 (1H, d, J=8.0 Hz, Hₐ-1″), 3.92 (1H, d, J=8.0 Hz, H_{b}-1″), 2.70 (3H, d, J=4.7 Hz, CH₃). FAB HR-MS: Theoretical Mass (C19H19BrN6O4): 475.07293 (M+H), Measured Mass: 475.073360.

### Example 6

### (1S,3R,4R,7S)-7-Benzyloxy-1-benzoyloxymethyl-3-(N6-(3-chlorobenzyl)-2-chloro-adenin-9-yl)-2,5-dioxabicyclo[2:2:1]heptane, 5a

1,2-O-acetyl-3-O-benzyl-4-C-methanesulfonyloxymethyl-5-O-methanesulfonyloxymethyl-α,β-D-ribofuranose (2, J. Org. Chem., 2001, 66, 8504-8512) (8.6 g, 16.8 mmol), 2,6-dichloropurine (4.0 g, 21.0 mmol) and N,O-bis(trimethylsilyl)acetamide (7.7 g, 37.8 mmol) was dissolved in anhydrous 1,2-dichloroethane (150 mL) and added trimethylsilyl trifluoromethanesulfonate (7.5 g, 33.6 mmol). The mixture was stirred at 70° C. for 5 h and then diluted with EtOAc (100 mL) and quenched with a solution of sat. aq. NaHCO₃ (150 mL). The phases were separated and the aq. phase extracted with EtOAc. The combined organic phase was dried over Na₂SO₄, filtered and the solvent removed in vacuo to give intermediate 3 as a yellow oil. A mixture of 3 (2.0 g, 3.13 mmol), triethylamine (10 mL) and 3-chlorobenzylamine (0.53 g, 3.75 mmol) in MeOH (50 mL) was stirred at room temperature for 16 h. A 1M aq. solution of NaOH (5 mL) was added and stirring continued for 2 h. The mixture was neutralized by adding a 1M aq. solution of HCl and then added brine (50 mL) and H₂O (50 mL). The phases were separated and the aq. phase extracted three times with DCM. The combined organic phase was dried over Na₂SO₄, filtered and the solvent removed in vacuo to give intermediate 4a as solid foam (ESI-MS m/z 606.2 (MH⁺)). All of 4a was dissolved in anhydrous DMSO (50 mL), added NaOBz (1.35 g, 9.4 mmol) and stirred at 100° C. for 16 h. The mixture was cooled to room temperature and added EtOAc (50 mL), brine (50 mL) and H₂O (50 mL). The phases were separated and the aq. phase was extracted twice with EtOAc. The combined organic phase was dried over Na₂SO₄, filtered and the solvent removed in vacuo to give 5a a yellow oil. 1.5 g. ESI-MS m/z 632.3 (MH⁺).

### Example 7

### (1S,3R,4R,7S)-7-Benzyloxy-1-benzoyloxymethyl-3-(N6-(3-bromobenzyl)-2-chloro-adenin-9-yl)-2,5-dioxabicyclo[2:2:1]heptane, 5b

Prepared by the same procedure as for 5a using 3 (2.0 g, 3.13 mmol) and 3-bromobenzylamine (0.70 g, 3.75 mmol). Gave 5b as an yellow oil. 1.5 g. ESI-MS m/z 676.1 (MH⁺).

### Example 8

### (1S,3R,4R,7S)-7-Benzyloxy-1-benzoyloxymethyl-3-(N6-(3-iodobenzyl)-2-chloro-adenin-9-yl)-2,5-dioxabicyclo[2:2:1]heptane, 5c

Prepared by the same procedure as for 5a using 3 (150 mg, 0.24 mmol) and 3-iodobenzylamine (66 mg, 0.28 mmol). Gave 5c as a yellow oil. 140 mg. ESI-MS m/z 724.2 (MH⁺).

### Example 9

### (1S,3R,4R,7S)-Benzyloxy-1-hydroxyoxymethyl-3-(N6-(3-chlorobenzyl)-2-chloro-adenin-9-yl)-2,5-dioxabicyclo[2:2:1]heptane, 6a

5a (1.5 g, 2.4 mmol) was dissolved in CH₃CN (50 mL) and added a 1M aq. solution of LiOH (20 mL) and stirring continued for 7 h. The mixture was neutralized by adding a 1M aq. solution of HCl and then added brine (50 mL). The phases were separated and the aq. phase extracted three times with DCM. The combined organic phase was dried over Na₂SO₄, filtered and the solvent removed in vacuo to give the crude product. Purification by general method E (50-100% EtOAc in heptane) gave 6a as yellow oil. 1.3 g. ESI-MS m/z 528.2 (MH⁺).

### Example 10

### (1S,3R,4R,7S)-7-Benzyloxy-1-hydroxyoxymethyl-3-(N6-(3-bromobenzyl)-2-chloro-adenin-9-yl)-2,5-dioxabicyclo[2:2:1]heptane, 6b

Prepared from 5b (1.5 g, 2.2 mmol) using the same procedure as for 6b. Purification by general method E (50-100% EtOAc in heptane) gave 6a as yellow oil. 1.45 g. ESI-MS m/z 572.2 (MH⁺).

### Example 11

### (1S,3R,4R,7S)-7-Benzyloxy-1-hydroxyoxymethyl-3-(N6-(3-iodobenzyl)-2-chloro-adenin-9-yl)-2,5-dioxabicyclo[2:2:1]heptane, 6c

Prepared from 5c (140 mg, 0.22 mmol) using the same procedure as for 6a. Purification by general method F gave 6c as white solid. 80 mg. ESI-MS m/z 620.1 (MH⁺).

### Example 12

### (1S,3R,4R,7S)-7-Hydroxy-1-hydroxymethyl-3-(N6-(3-iodobenzyl)-2-chloro-adenin-9-yl)-2,5-dioxabicyclo[2:2:1]heptane (SPN0174)

5c was subjected to general method D followed by hydrolysis with LION as described for the preparation of 6a-6c. Purification by general method F gave 20 mg of the target compound as a white solid. ¹H-NMR (Methanol-d₄, 300 MHz) δ 8.18 (1H, s, H-8), 7.77 (1H, br s, 3-I-Ph), 7.58 (1H, d, J=7.9 Hz, 3-I-Ph), 7.37 (1H, d, J=7.7, Hz, 3-I-Ph), 7.11 (1H, t, J=7.8 Hz, 3-I-Ph), 5.94 (1H, s, H-1′), 4.69 (2H, br 5, 3-I-PhCH₂), 4.49 (1H, s, H-2′ or H-3′), 4.32 (1H, s, H-2′ or H-3′), 4.05 (1H, d, J=8.0 Hz, Hₐ-1″), 3.93 (2H, br s, H-5′), 3.88 (1H, d, J=8.0 Hz, H_{b}-1″). FAB HR-MS: Theoretical Mass (C18H17ClN5O4): 530.0092 (M+H), Measured Mass: 530.010193.

### Example 13

### (1R,3R,4R,7S)-(7-hydroxy-3-(N6-(3-iodobenzyl)-2-chloro-adenin-9-yl)-2,5-dioxabicyclo[2:2:1]hept-1-yl)-carboxylic acid (SPN0176)

A solution of SPN0174 (50 mg, 0.094 mmol), diacetoxyiodobenzene (71 mg, 0.22 mmol) and 2,2,6,6-tetramethylpiperidine-1-oxyl (5 mg, 0.03 mmol) was stirred at 40° C. for 3 h after which the solvent as removed in vacuo. The residue was purified by general method F to give 40 mg of the target compound as a white solid. ¹H-NMR (DMSO-d₆, 300 MHz) δ 9.01 (1H, br t, J=5.8 Hz, CH₂NH), 8.21 (1H, s, H-8), 7.74 (1H, s, 3-I-Ph), 7.59 (1H, d, J=7.9 Hz, 3-I-Ph), 7.33 (1H, d, J=7.4 Hz, 3-I-Ph), 7.12 (1H, t, J=7.8 Hz, 3-I-Ph), 5.90 (1H, s, H-1′), 4.60 (2H, br s, 3-I-PhCH₂), 4.58 (1H, s, H-2′ or H-3′), 4.52 (1H, br s, H-2′ or H-3′), 4.26 (1H, d, J=8.0 Hz, Hₐ-1″), 3.97 (1H, d, J=8.0 Hz, H_{b}-1″). FAB HR-MS: Theoretical Mass (C18H15ClN5O5): 543.98847 (M+H), Measured Mass: 543.98853.

### Example 14

### (1R,3R,4R,7S)-(7-hydroxy-3-(N6-(3-iodobenzyl)-2-chloro-adenin-9-yl)-2,5-dioxabicyclo[2:2:1]hept-1-yl)-N-methylcarboxamide (SPN0175)

6c was subjected to general method A followed by general method D to give the crude product. Purification by general method F gave 31 mg of the target compound as a white solid. ¹H-NMR (DMSO-d₆, 300 MHz) δ 9.00 (1H, br t, J=5.4 Hz CH₂NH), 8.40 (1H, s, H-8), 8.20 (1H, br s, NHMe), 7.74 (1H, s, 3-I-Ph), 7.59 (1H, d, J=7.9 Hz, 3-I-Ph), 7.34 (1H, d, J=7.4 Hz, 3-I-Ph), 7.13 (1H, t, J=7.8 Hz, 3-I-Ph), 6.10 (1H, br s, OH), 5.95 (1H, s, H-1′), 4.60-4.51 (4H, m, 3-I-PhCH₂, H-2′ and H-3′), 4.32 (1H, d, J=8.1 Hz, Hₐ-1″), 3.94 (1H, d, J=8.1 Hz, H_{b}-1″), 2.69 (3H, d, J=4.5 Hz, CH₃). FAB HR-MS: Theoretical Mass (C19H18ClN6O4): 557.0201 (M+H), Measured Mass: 557.01964.

### Example 15

### (1R,3R,4R,7S)-(7-hydroxy-3-(N6-(3-chlorobenzyl)-2-chloro-adenin-9-yl)-2,5-dioxabicyclo[2:2:1]hept-1-yl)-N-methylcarboxamide (SPN0197)

6a was subjected to general method A followed by general method D to give the crude product. Purification by general method F gave 130 mg of the target compound as a white solid. ¹H-NMR (DMSO-d₆, 300 MHz) δ 9.01 (1H, br t, J=5.8 Hz, CH₂NH), 8.40 (1H, s, H-8), 8.19 (1H, br d, J=4.2 Hz, NHMe), 7.40-7.29 (4H, m, 3-Cl-Ph), 6.00 (1H, br d, J=4.2 Hz, OH), 5.97 (1H, s, H-1′), 4.64 (2H, br d, J=5.8 Hz, 3-Cl-PhCH₂), 4.57 (1H, s, H-2′), 4.50 (1H, br s, H-3′), 4.33 (1H, d, J=8.0 Hz, Hₐ-1″), 3.91 (1H, d, J=8.0 Hz, H_{b}-1″), 2.7 (3H, d, J=4.6 Hz, CH₃). FAB HR-MS: Theoretical Mass (C19H18Cl2N6O4): 465.08448 (M+H), Measured Mass: 465.084007.

### Example 16

### (1R,3R,4R,7S)-(7-hydroxy-3-(N6-(3-bromobenzyl)-2-chloro-adenin-9-yl)-2,5-dioxabicyclo[2:2:1]hept-1-yl)-N-methylcarboxamide (SPN0196)

6b was subjected to general method A followed by general method D to give the crude product. Purification by general method F gave 125 mg of the target compound as a white solid. ¹H-NMR (DMSO-d₆, 300 MHz) δ 9.01 (1H, br t, J=6.0 Hz, CH₂NH), 8.40 (1H, s, H-8), 8.18 (1H, br d, J=4.2 Hz, NHMe), 7.55 (1H, br s, 3-Br-Ph), 7.44 (1H, d, J=7.6 Hz, 3-Br-Ph), 7.34 (1H, br d, J=7.5 Hz, 3-Br-Ph), 7.28 (1H, t, J=7.6 Hz, 3-Br-Ph), 6.01 (1H, br s, OH), 5.97 (1H, s, H-1′), 4.65 (2H, br d, J=6.0 Hz, 3-Br-PhCH₂), 4.57 (1H, s, H-2′), 4.51 (1H, br s, H-3′), 4.33 (1H, d, J=8.0 Hz, Hₐ-1″), 3.91 (1H, d, J=8.0 Hz, H_{b}-1″), 2.71 (3H, d, J=4.6 Hz, CH₃). FAB HR-MS: Theoretical Mass (C19H18BrClN6O4): 509.03396 (M+H), Measured Mass: 509.033374.

### Example 17

### 1-(2-Trifluoroacetylamino-3-O-benzyl-4-C-methanesulfonyloxymethyl-5-O-methanesulfonyl-2-deoxy-β-D-ribofuranosyl)-thymine (8)

To a stirred solution of 1-(2-azido-3-O-benzyl-4-C-methanesulfonyloxymethyl-5-O-methanesulfonyl-2-deoxy-β-D-ribofuranosyl)-thymine (7, Org. Biomol. Chem., 2003, 655-663) (4.6 g, 8.2 mmol) in THF (200 mL) and H₂O (0.2 mL) was added a solution of PMe₃ in THF (1M, 16.4 mL, 16.4 mmol) and the mixture was stirred at room temperature 30 min. The solution was cooled to 0° C. and trifluoroacetic acid anhydride was added drop-wise over 10 min and stirring was continued at room temperature for 3 h. The reaction was quenched by the addition of H₂O (200 mL) and then neutralized using solid NaHCO₃. The mixture was extracted with DCM (2×200 mL) and the combined organic phase was dried over Na₂SO₄, filtered, and evaporated to a white foam. Purification by general method E (20-100% EtOAc in n-heptane) gave 8 (4.1 g, 79%) as a white solid foam. ESI-MS m/z 630.1 [M+H]⁺

### Example 18

### 1-(2-Trifluoroacetylamino-3-O-benzyl-4-O-methanesulfonyloxymethyl-5-O-methanesulfonyl-2-deoxy-β-D-ribofuranosyl)-N⁶-benzoyladenine (9)

To a stirred suspension of N⁶-benzoyladenine (1.87 g, 7.82 mmol) in DCE (25 mL) was added BSA (3.9 mL, 15.7 mmol) and the mixture was stirred at reflux for 1 h to give a clear solution. Nucleoside 8 (1.64 g, 2.61 mmol) in DCE (20 mL) was added followed by TMSOTf (2.4 mL, 13.1 mmol) and stirring continued at reflux. After 16 h the reaction mixture was allowed to reach room temperature and quenched by the addition of saturated aq. NaHCO₃ (50 mL). The phases were separated and the aq. phase was extrated with DCM (2×75 mL). The combined organic phase was washed with 1M aq. HCl (100 mL), dried over Na₂SO₄, filtered, and evaporated to a yellow solid. Purification by general method E (0-100% EtOAc in n-heptane) gave 9 (1.10 g, 57%) as a white solid foam. ESI-MS m/z 742.2 [M+H]⁺

### Example 19

### (1R,3R,4R,7S)-7-Benzyloxy-1-methanesulfonyloxymethyl-3-(N⁶-benzoyladenine-9-yl)-2-oxa-5-aza-bicyclo[2:2:1]heptane (10)

To a solution of nucleoside 9 (1.0 g, 1.35 mmol) in THF (50 mL) was added 1M aq. LiOH (50 mL) and the mixture was stirred vigorously at room temperature for 16 h. 1M aq. HCl was added until pH 8 and the mixture was extracted with DCM ((2×100 mL). The combined organic phase was washed with saturated aq. NaHCO₃ (100 mL), dried over Na₂SO₄, filtered, and evaporated to a white solid foam (0.73 g, 99%). ESI-MS m/z 551.1 [M+H]⁺

### Example 20

### (1R,3R,4R,7S)-7-Benzyloxy-5-tert-butoxycarbonyl-1-hydroxymethyl-3-(adenine-9-yl)-2-oxa-5-aza-bicyclo[2:2:1]heptane (11)

To a solution of 10 (500 mg, 0.91 mmol) in DCM (20 mL) and triethylamine (3 mL) was added Boc₂O (418 μL, 1.82 mmol) and the solution was stirred at room temperature for 2 h. The solution was washed with saturated aq. NaHCO₃ (20 mL), dried over Na₂SO₄, filtered, and evaporated to a yellow oil (ESI-MS m/z 651.2 [M+H]⁺). The oil was redissolved in DMSO (10 mL) and added NaOBz (525 mg, 3.6 mmol) and the mixture was stirred at 90° C. for 2 h. H₂O (20 mL) was added and the mixture was extracted with DCM (3×50 mL). The combined organic phase was washed with brine (2×50 mL), dried over Na₂SO₄, filtered, and evaporated to a yellow solid foam (ESI-MS m/z 677.1 [M+H]⁺). The solid was dissolved in saturated NH₃ in methanol (30 mL) and stirred at room temperature for 16 h. The solvent was removed in vacuo and purification by general method E (0-5% MeOH in DCM) gave 11 (215 mg, 50%) as a white solid foam. ESI-MS m/z 469.1 [M+H]⁺

### Example 21

### (1R,3R,4R,7S)-(7-hydroxy-3-(adenin-9-yl)-2-oxa-5-azabicyclo[2:2:1]hept-1-yl)-N-methylcarboxamide (SPN0238)

11 was subjected to general method A followed by general method B to give the crude product. Purification by general method F gave 39 mg of the target compound as a white solid. ¹H-NMR (DMSO-d₆, 300 MHz) δ 8.34 (1H, s, H-2), 8.17 (1H, s, H-8), 8.16 (1H, br s, NHMe), 7.32 (2H, s, NH₂), 5.88 (1H, s, H-1′), 4.32 (1H, s, H-3′), 3.69 (1H, s, H-2′), 3.50 (1H, d, J=10.3 Hz, Hₐ-1″), 2.85 (1H, d, J=10.3 Hz, H_{b}-1″), 2.68 (3H, d, J=4.6 Hz, CH₃). CI HR-MS: Theoretical Mass (C12H15N7O3): 306.13146 (M+H), Measured Mass: 306.131584.

### Example 22

### (1S,3R,4R,7S)-7-Hydroxy-1-hydroxymethyl-3-(N6-(3-iodobenzyl)-adenin-9-yl)-2-oxa-5-azabicyclo[2:2:1]heptane (SPN0234)

11 was subjected to general method B followed by general method C to give an yellow oil. This was treated with trifluoroacetic acid for 1 h and the solvent was removed in vacuo to give the crude product. Purification by general method F gave 16 mg of the target compound as a white solid. ¹H-NMR (DMSO-d₆, 300 MHz) δ 8.43 (1H, br s, CH₂NH), 8.26 (1H, s, H-2), 8.21 (1H, s, H-8), 7.73 (1H, br s, 3-I-Ph), 7.57 (1H, d, J=7.7 Hz, 3-I-Ph), 7.35 (1H, d, J=7.6, Hz, 3-I-Ph), 7.11 (1H, t, J=7.7 Hz, 3-I-Ph), 5.83 (1H, s, H-1′), 5.35 (1H, br s, OH), 4.97 (1H, br s, OH), 4.67 (2H, br s, 3-I-PhCH₂), 4.10 (1H, s, H-3′), 3.74 (2H, br s, H-5′), 3.54 (1H, s, H-2′), 2.99 (1H, d, J=10.0 Hz, Hₐ-1″), 2.74 (1H, d, J=10.0 Hz, H_{b}-1″). FAB HR-MS: Theoretical Mass (C18H19IN6O3): 495.06416 (M+H), Measured Mass: 495.063836.

### Example 23

### (1R,3R,4R,7S)-(7-hydroxy-3-(N6-(3-iodobenzyl)-adenin-9-yl)-2-oxa-5-azabicyclo[2:2:1]hept-1-yl)-N-methylcarboxamide (SPN0235)

11 was subjected to general method C followed by general method A and general method D to give the crude product. Purification by general method F gave 14 mg of the target compound as a white solid. ¹H-NMR (DMSO-d₆, 300 MHz) δ 8.48 (1H, br s, CH₂NH), 8.38 (1H, s, H-2), 8.24 (1H, s, H-8), 8.10 (1H, br s, NHMe), 7.73 (1H, s, 3-I-Ph), 7.60 (1H, d, J=7.9 Hz, 3-I-Ph), 7.38 (1H, d, J=7.6 Hz, 3-I-Ph), 7.11 (1H, t, J=7.7 Hz, 3-I-Ph), 5.92 (1H, s, H-1′), 5.67 (1H, br s, OH), 4.68 (2H, br s, 3-I-PhCH₂), 4.36 (1H, s, H-3′), 3.77 (1H, s, H-2′), 3.52 (1H, d, J=10.4 Hz, Hₐ-1″), 2.89 (1H, d, J=10.4 Hz, H_{b}-1″), 2.68 (3H, d, J=4.6 Hz, CH₃). FAB HR-MS: Theoretical Mass (C19H20IN7O3): 522.07506 (M+H), Measured Mass: 522.073557.

### Example 24

### 9-(2-O-Acetyl-3-azido-4-C-methanesulfonyloxymethyl-5-O-methanesulfonyl-3-deoxy-β-D-ribofuranosyl)-N6-benzoyladenine (13)

9-(2-O-Acetyl-3-O-benzyl-4-C-methanesulfonyloxymethyl-5-O-methanesulfonyl-β-D-xylofuranosyl)-N6-benzoyladenine (12, J. Am. Chem. Soc., 2002, 124, 2164-2176) (9.7 g, 14.1 mmol) was dissolved in acetic acid (200 mL) and added ammonium formate (2.7 g, 42.2 mmol) and Pd(OH)₂ (20% on carbon, 0.3 g). The mixture was stirred at 75° C. for 6 h and then at 50° C. for 16 h. Cooled to room temperature and filtered through a pad of Celite and the solvent was removed in vacuo. The residue was redissolved in EtOAc (300 mL) and washed with H₂O (100 mL), saturated aq. NaHCO₃ (100 mL), dried over Na₂SO₄, filtered, and evaporated to a white solid foam (6.3 g, 74%). 4.5 g (7.5 mmol) of this intermediate was dissolved in anhydrous DCM (150 mL) and added pyridine (10 mL). The solution was cooled to 0° C. and added Tf₂O (1.9 mL, 11 mmol) drop-wise over 10 min. The mixture was stirred at 0° C. for 2 h and then diluted with DCM (150 mL), washed with brine (2×100 mL), dried over Na₂SO₄, filtered, and evaporated to a dark brown solid foam. The material was redissolved in anhydrous DMF (100 mL) and added sodium azide (1.3 g, 20 mmol). The mixture was stirred at 60° C. for 2 h and the solvent was removed in vacuo to give a brown oil. Purification by general method E (50-100% EtOAc in n-heptane) gave 13 (1.65 g) as a light brown solid foam. ESI-MS m/z 625.2 [M+H]⁺.

### Example 25

### (1R,3R,4R,7S)-7-Azido-1-hydroxymethyl-3-(adenin-9-yl)-2,5-dioxabicyclo[2:2:1]heptane (14)

Compound 13 (1.65 g) was ring-closed and the mesylate group removed using the same procedure as for the conversion of 9 to 11 (Example 19 and 20, excluding treatment with Boc₂O). The crude product was partitioned between H₂O (30 mL) and DCM (30 mL). A brown precipitate was filtered off and dried under vacuum for 16 h. The phases were separated. The Organic phase was extracted with H₂O and the combined aq. phase was freeze-dried and combined with the dried precipitate to give a total of 500 mg of the target product 14. ESI-MS m/z 305.1 [M+H]⁺.

### Example 26

### (1R,3R,4R,7S)-7-Azido-1-hydroxymethyl-3-(N6-(3-iodobenzyl)-adenin-9-yl)-2,5-dioxabicyclo[2:2:1]heptane (SPN0190)

14 was subjected to general method C to give the crude product. Purification by general method F gave 43 mg of the target compound as a white solid. ¹H-NMR (DMSO-d₆, 300 MHz) δ 8.31 (1H, s, H-2), 8.10 (1H, s, H-8), 7.80 (1H, br s, 3-I-Ph), 7.66 (1H, d, J=7.9 Hz, 3-I-Ph), 7.38 (1H, d, J=7.7, Hz, 3-I-Ph), 7.14 (1H, t, J=7.8 Hz, 3-I-Ph), 5.89 (1H, s, H-1′), 5.25 (1H, d, J=14.9 Hz, 3-I-PhCH₂), 5.16 (1H, d, J=14.9 Hz, 3-I-PhCH₂), 4.75 (1H, s, H-2′ or H-3′), 4.47 (1H, s, H-2′ or H-3′), 3.88 (1H, d, J=8.5 Hz, Hₐ-1″), 3.79 (2H, br s, H-5′), 3.77 (1H, d, J=8.5 Hz H_{b}-1″). FAB HR-MS: Theoretical Mass (C18H17IN8O3): 521.05466 (M+H), Measured Mass: 521.054352.

### Example 27

### (1R,3R,4R,7S)-7-Amino-hydroxymethyl-3-(N6-(3-iodobenzyl)-adenin-9-yl)-2,5-dioxabicyclo[2:2:1]heptane (SPN0191)

To a solution of SPN0190 (20 mg, 0.038 mmol) in THF (1 mL) was added 2M aq. NaOH (0.5 mL) and a 1M solution of PMe₃ in THF (152 μL, 0.152 mmol). The mixture was stirred at room temperature for 16 h and then diluted with brine and extracted with DCM (2×10 mL). The combined organic phases was evaporated in vacuo to give a clear oil. The oil was triturated with 1M HCl in Et₂O to give 20 mg of the target product as a white solid. ¹H-NMR (DMSO-d₆, 300 MHz) δ 8.86 (2H, br s, CH₂NH₂⁺), 8.44 (1H, s, H-2), 8.32 (1H, s, H-8), 7.75 (1H, br s, 3-I-Ph), 7.61 (1H, d, J=7.9 Hz, 3-I-Ph), 7.40 (1H, d, J=7.7, Hz, 3-I-Ph), 7.15 (1H, t, J=7.8 Hz, 3-I-Ph), 6.13 (1H, s, H-1′), 4.74 (2H, br s, 3-I-PhCH₂), 5.00 (1H, s, H-2′), 4.75 (1H, s, H-2′ or H-3′), 4.20 (1H, s, H-3′), 4.24 (1H, d, J=8.5 Hz, Hₐ-1″), 4.13 (2H, br s, H-5′), 3.95 (1H, d, J=8.5 Hz H_{b}-1″). FAB HR-MS: Theoretical Mass (C18H19IN6O3): 495.06416 (M+H), Measured Mass: 495.064464.

### Example 28

### (1S,3R,4R,7S)-(7-azido-3-(N6-(3-iodobenzyl)-adenin-9-yl)-2,5-dioxabicyclo[2:2:1]hept-1-yl)-N-methylcarboxamide (SPN0192)

14 was subjected to general method A followed by general method C to give the crude product. Purification by general method F gave 52 mg of the target compound as a white solid. ¹H-NMR (DMSO-d₆, 300 MHz) δ 8.56 (1H, br s, CH₂NH), 8.47 (1H, s, H-2), 8.33 (1H, m, NHMe), 8.25 (1H, s, H-8), 7.72 (1H, br s, 3-I-Ph), 7.57 (1H, d, J=7.9 Hz, 3-I-Ph), 7.34 (1H, d, J=7.7, Hz, 3-I-Ph), 7.11 (1H, t, J=7.7 Hz, 3-I-Ph), 6.09 (1H, s, H-1′), 4.96 (1H, s, H-2′ or H-3′), 4.91 (1H, s, H-2′ or H-3′), 4.66 (2H, br s, 3-I-PhCH₂), 4.21 (1H, d, J=8.4 Hz, Hₐ-1″), 4.00 (1H, d, J=8.4 Hz, H_{b}-1″), 2.70 (3H, d, J=4.7 Hz, CH₃). FAB HR-MS: Theoretical Mass (C19H18IN9O3): 548.06556 (M+H), Measured Mass: 548.065848.

### Example 29

(1S,3R,4R,7S)-(7-amino-3-(N6-(3-iodobenzyl)-adenin-9-yl)-2,5-dioxabicyclo[2:2:1]hept-1-yl)-N-methylcarboxamide (SPN0195): Prepared from SPN0192 using the same procedure as for SPN0191. Trituration with 1M HCl in Et₂O gave 26 mg of the target product as a white solid. ¹H-NMR (DMSO-d₆, 300 MHz) δ 8.88 (2H, br s, CH₂NH₂⁺), 8.68 (1H, s, H-2), 8.47 (1H, m, NHMe), 8.36 (1H, s, H-8), 7.77 (1H, br s, 3-I-Ph), 7.62 (1H, d, J=7.6 Hz, 3-I-Ph), 7.40 (1H, d, J=7.5, Hz, 3-I-Ph), 7.13 (1H, t, J=7.7 Hz, 3-I-Ph), 6.21 (1H, s, H-1′), 5.35 (2H, br s, 3-I-PhCH₂), 5.21 (1H, s, H-2′), 4.77 (1H, s, H-3′), 4.46 (1H, d, J=9.4 Hz, Hₐ-1″), 4.13 (1H, d, J=9.4 Hz, H_{b}-1″), 2.70 (3H, d, J=4.7 Hz, CH₃). FAB HR-MS: Theoretical Mass (C19H20IN7O3): 522.07506 (M+H), Measured Mass: 522.075562.

### Example 30

### (1R,3R,4S,7R)-7-Benzyloxy-1-hydroxymethyl-5-trifluoroacetyl-3-(adenin-9-yl)-2-oxa-5-azabicyclo[2:2:1]heptane (16)

(1R,3R,4S,7R)-7-Benzyloxy-1-hydroxymethyl -5-trifluoroacetyl-3-(adenin-9-yl)-2-oxa-5-azabicyclo[2:2:1]heptane (15, WO03095476) (5.51 g, 10 mmol) was dissolved in anhydrous DCM (150 mL) and triethylamine (10 mL) and cooled to 0° C. Trifluoroacetic acid anhydride (2.1 mL, 15 mmol) was added dropwise over 5 min and stirring was continued for 30 min at room temperature. The solution was diluted with DCM (150 mL), washed with saturated aq. NaHCO₃ (2×100 mL), dried over Na₂SO₄, filtered, and evaporated to an off-white solid foam. The mesylate group was removed using the same procedure as for the conversion of 9 to 11 (Example 19 and 20, excluding treatment with Boc₂O). The resulting crude product was purified by general method E (0-8% MeOH in EtOAc) to give 16 as a colorless oil (3.31 g, 71%). ESI-MS m/z 465.1 [M+H]⁺.

### Example 31

### (1S,3R,4S,7R)-7-hydroxy-5-trifluoroacetyl-3-(adenin-9-yl)-2-oxa-5-azabicyclo-[2:2:1]hept-1-yl)-N-methylcarboxamide (17)

16 was subjected to general method A and purified by general method E (0-10% MeOH in EtOAc). General method B was applied to give 17 which was used without further purification. ESI-MS m/z 402.2 [M+H]⁺.

### Example 32

### (1S,3R,4S,7R)-7-hydroxy-3-(adenin-9-yl)-2-oxa-5-azabicyclo[2:2:1]hept-1-yl)-N-methylcarboxamide (SPN0241)

A solution of 17 (100 mg, 0.25 mmol) in CH₃CN (2 mL) and conc. NH₄OH (2 mL) was stirred at 50° C. for 1 h and the solvents were removed in vacuo to give the crude product. Purification by general method F gave 18 mg of the target compound as a white solid. ¹H-NMR (DMSO-d₆, 300 MHz) δ 8.48 (1H, s, H-2), 8.16 (1H, s, H-8), 7.92 (1H, br s, NHMe), 7.25 (2H, s, NH₂), 6.45 (1H, s, H-1′), 5.88 (1H, br s, OH), 4.46 (1H, s, H-3′), 3.61 (1H, s, H-2′), 3.54 (1H, d, J=10.5 Hz, Hₐ-1″), 3.10 (1H, d, J=10.5 Hz, H_{b}-1″), 2.64 (3H, d, J=4.5 Hz, CH₃). CI HR-MS: Theoretical Mass (C12H15N7O3): 306.13146 (M+H), Measured Mass: 306.131056.

### Example 33

### (1R,3R,4S,7R)-7-Hydroxy-1-hydroxymethyl-3-(N6-(3-iodobenzyl)-adenin-9-yl)-2-oxa-5-azabicyclo[2:2:1]heptane (SPN0240)

16 was subjected to general method B followed by general method C to give the crude product. Purification by general method F gave 50 mg of the target compound as a white solid. ¹H-NMR (DMSO-d₆, 300 MHz) δ 8.48 (1H, s, H-8), 8.38 (1H, br s, CH₂NH), 8.19 (1H, s, H-2), 7.73 (1H, br s, 3-I-Ph), 7.57 (1H, d, J=7.6 Hz, 3-I-Ph), 7.35 (1H, d, J=7.3, Hz, 3-I-Ph), 7.11 (1H, t, J=7.8 Hz, 3-I-Ph), 6.30 (1H, s, H-1′), 5.59 (1H, br s, OH), 4.75 (1H, br s, OH), 4.68 (2H, br s, 3-I-PhCH₂), 4.25 (1H, s, H-3′), 3.68 (2H, br s, H-5′), 3.41 (1H, s, H-2′), 3.11 (1H, d, J=10.3 Hz, Hₐ-1″), 2.93 (1H, d, J=10.3 Hz, H_{b}-1″). Theoretical Mass (C18H19IN6O3): 495.06416 (M+H), Measured Mass: 495.063372.

### Example 34

### (1S,3R,4S,7R)-(7-hydroxy-3-(N6-(3-iodobenzyl)-adenin-9-yl)-2-oxa-5-azabicyclo[2:2:1]hept-1-yl)-N-methylcarboxamide (SPN0242)

17 was subjected to general method C to give the crude product. Purification by general method F gave 28 mg of the target compound as a white solid. ¹H-NMR (CDCl₃, 300 MHz) δ 8.68 (1H, s, H-2), 8.38 (1H, s, H-8), 7.74 (1H, s, CH₂NH), 7.62 (1H, d, J=7.2 Hz, 3-I-Ph), 7.33 (1H, d, J=7.4 Hz, 3-I-Ph), 7.26 (1H, s, 3-I-Ph), 7.06 (1H, t, J=7.9 Hz, 3-I-Ph), 6.51 (1H, s, H-1′), 6.50 (1H, br s, NHMe), 6.26 (1H, br s, OH), 4.86 (2H, br s, 3-I-PhCH₂), 4.66 (1H, s, H-3′), 3.83 (1H, s, H-2′), 3.79 (1H, d, J=10.9 Hz, Hₐ-1″), 3.17 (1H, d, J=10.9 Hz, H_{b}-1″), 2.86 (3H, d, J=5.0 Hz, CH₃). CI HR-MS: Theoretical Mass (C19H20IN7O3): 522.07506 (M+H), Measured Mass: 522.07456.

### Example 35

### (1S,3R,4R,7R)-7-Benzyloxy-1-hydroxymethyl-3-(adenin-9-yl)-2,5-dioxabicyclo[2:2:1]heptane (18)

9-(2-O-Acetyl-3-O-benzyl-4-C-methanesulfonyloxymethyl-5-O-methanesulfonyl-β-D-xylofuranosyl)-N6-benzoyladenine (12, J. Am. Chem. Soc., 2002, 124, 2164-2176) (8.0 g, 12.4 mmol) was ring-closed and the mesylate group removed using the same procedure as for the conversion of 9 to 11 (Example 19 and 20, excluding treatment with Boc₂O) to give 18 as a white solid foam (5.7 g, 97%). ESI-MS m/z 474.2 [M+H]⁺.

### Example 36

### (1R,3R,4R,7R)-(7-hydroxy-3-(adenin-9-yl)-2,5-dioxabicyclo[2:2:1]hept-1-yl)-N-methylcarboxamide (SPN0199)

18 was subjected to general method A followed by general method B to give the crude product. Purification by general method F gave 70 mg of the target compound as a white solid. ¹H-NMR (DMSO-d₆, 300 MHz) δ 8.43 (1H, s, H-2), 8.15 (1H, s, H-8), 8.11 (1H, q, J=4.7 Hz, NHMe), 7.25 (2H, s, NH₂), 6.20 (1H, br s, OH), 6.16 (1H, s, H-1′), 4.63 (1H, d, J=2.1 Hz, H-2′), 4.44 (1H, d, J=2.1 Hz, H-3′), 4.25 (1H, d, J=8.2 Hz, Hₐ-1″), 3.95 (1H, d, J=8.2 Hz, H_{b}-1″), 2.71 (3H, d, J=4.7 Hz, CH₃). CI HR-MS: Theoretical Mass (C12H14N6O4): 307.11547 (M+H), Measured Mass: 307.1150129.

### Example 37

### (1S,3R,4R,7R)-7-Hydroxy-1-hydroxymethyl -3-(N6-(3-iodobenzyl)-adenin-9-yl)-2,5-dioxabicyclo[2:2:1]heptane (SPN0243)

18 was subjected to general method B followed by general method C to give the crude product. Purification by general method F gave 50 mg of the target compound as a white solid. ¹H-NMR (DMSO-d₆, 300 MHz) δ 8.37 (1H, br s, CH₂NH), 8.23 (1H, s, H-2), 8.22 (1H, s, H-8), 7.72 (1H, br s, 3-I-Ph), 7.59 (1H, d, J=7.6 Hz, 3-I-Ph), 7.34 (1H, d, J=7.4, Hz, 3-I-Ph), 7.10 (1H, t, J=7.7 Hz, 3-I-Ph), 6.04 (1H, s, H-1′), 5.84 (1H, br s, OH), 5.00 (1H, br s, OH), 4.67 (2H, br s, 3-I-PhCH₂), 4.49 (1H, br s, H-2′ or H-3′), 4.19 (1H, br s, H-2′ or H-3′), 4.07 (1H, d, J=8.1 Hz, Hₐ-1″), 3.91-3.79 (3H, m, H-5′, H_{b}-1″). Theoretical Mass (C18H18IN5O4): 496.04817 (M+H), Measured Mass: 496.04839.

### Example 38

### (1R,3R,4R,7R)-(7-hydroxy-3-(N6-(3-iodobenzyl)-adenin-9-yl)-2,5-dioxabicyclo[2:2:1]hept-1-yl)-N-methylcarboxamide (SPN0170)

SPN0199 was subjected to general method C to give the crude product. Purification by general method F gave 115 mg of the target compound as a white solid. ¹H-NMR (DMSO-d₆, 300 MHz) δ 8.48 (1H, s, H-2), 8.42 (1H, br s, CH₂NH), 8.22 (1H, s, H-8), 8.13 (1H, m, NHMe), 7.72 (1H, br s, 3-I-Ph), 7.56 (1H, d, J=7.9 Hz, 3-I-Ph), 7.34 (1H, d, J=7.7, Hz, 3-I-Ph), 7.10 (1H, t, J=7.8 Hz, 3-I-Ph), 6.19 (1H, br s, OH), 6.16 (1H, s, H-1′), 4.65 (2H, br s, 3-I-PhCH₂), 4.65 (1H, d, J=2.0 Hz, H-2′), 4.44 (1H, d, J=2.0 Hz, H-3′), 4.28 (1H, d, J=8.2 Hz, Hₐ-1″), 3.95 (1H, d, J=8.2 Hz, H_{b}-1″), 2.71 (3H, d, J=4.4 Hz, CH₃). FAB HR-MS: Theoretical Mass (C19H19IN6O4): 523.05907 (M+H), Measured Mass: 523.05852.

### Example 39

### (1R,3R,4R,7R)-(7-hydroxy-3-(N6-(3-chlorobenzyl)-adenin-9-yl)-2,5-dioxabicyclo[2:2:1]hept-1-yl)-N-methylcarboxamide (SPN0200)

SPN0199 was subjected to general method C to give the crude product. Purification by general method F gave 95 mg of the target compound as a white solid. ¹H-NMR (DMSO-d₆, 300 MHz) δ 8.48 (1H, s, H-2), 8.44 (1H, br s, CH₂NH), 8.23 (1H, s, H-8), 8.12 (1H, m, NHMe), 7.38-7.25 (4H, m, 3-Cl-Ph), 6.19 (1H, br s, OH), 6.16 (1H, s, H-1′), 4.70 (2H, br s, 3-Cl-PhCH₂), 4.65 (1H, d, J=2.0 Hz, H-2′), 4.45 (1H, d, J=2.0 Hz, H-3′), 4.28 (1H, d, J=8.1 Hz, Hₐ-1″), 3.95 (1H, d, J=8.1 Hz, H_{b}-1″), 2.70 (3H, d, J=4.4 Hz, CH₃). FAB HR-MS: Theoretical Mass (C19H19ClN6O4): 431.12345 (M+H), Measured Mass: 431.122848.

### Example 40

### (1R,3R,4R,7R)-(7-hydroxy-3-(N6-(3-bromobenzyl)-adenin-9-yl)-2,5-dioxabicyclo[2:2:1]hept-1-yl)-N-methylcarboxamide (SPN0201)

SPN0199 was subjected to general method C to give the crude product. Purification by general method F gave 110 mg of the target compound as a white solid. ¹H-NMR (DMSO-d₆, 300 MHz) δ 8.48 (1H, s, H-2), 8.44 (1H, br s, CH₂NH), 8.23 (1H, s, H-8), 8.12 (1H, m, NHMe), 7.53 (1H, br s, 3-Br-Ph), 7.40 (1H, dt, J=7.8 and 1.3 Hz, 3-Br-Ph), 7.33 (1H, d, J=7.7 Hz, 3-Br-Ph), 7.27 (1H, t, J=7.8 Hz, 3-Br-Ph), 6.19 (1H, br s, OH), 6.16 (1H, s, H-1′), 4.70 (2H, br s, 3-Br-PhCH₂), 4.65 (1H, d, J=2.1 Hz, H-2′), 4.43 (1H, d, J=2.1 Hz, H-3′), 4.25 (1H, d, J=8.1 Hz, Hₐ-1″), 3.95 (1H, d, J=8.1 Hz, H_{b}-1″), 2.71 (3H, d, J=4.6 Hz, CH₃). FAB HR-MS: Theoretical Mass (C19H19BrN6O4): 475.07293 (M+H), Measured Mass: 475.072901.

### Example 41

### Adenosine A₃ Receptor Assays

AequoScreen™ (Euroscreen, Belgium) cell lines expressing the A₃ human recombinant receptor and the promiscuous G protein G_{α16} were used throughout the study. AequoScreen™ cells were cultured following recommended conditions for at least one week prior to the test. The day before the test, cells were harvested with PBS-EDTA, washed and re-suspended in BSA-DMEM12 (Dulbecco's Modified Eagles Medium—Ham's F12 with 0.1% BSA). Suspended cells were then incubated at room temperature with coelenterazine h overnight. For agonist and antagonist testing, 50 μl of the cell suspension were injected onto 50 μl of the test compound or control in 96-well plates, and the resulting emission of light measured for the determination of cell activation. After the first reading, and following an incubation time of 15-30 min, 100 μl of the reference agonist (IB-MECA) at a concentration equal to the EC80 of the day of the experiment were injected onto the cell suspension containing the test compounds. The resulting emission of light was measured for the determination of antagonistic effects. Light emission for agonist and antagonist tests was recorded using a Hamamatsu FDSS-6000 reader. For agonist data, percentages of activation were calculated on the basis of the activation (luminescence data) induced by the reference agonist at a saturating concentration (EC₁₀₀). For antagonist data, percentages of inhibition were calculated on the basis of the activation (luminescence data) induced by the reference agonist at a concentration equal to the EC₈₀. The test compounds were tested as duplicate determinations at 8 concentrations of 100, 10, 1, 0.1 μM and 10, 1, 0.1, and 0.01 nM for agonist activity and 50, 5, 0.5, 0.05 μM and 5, 0.5, 0.05, and 0.005 nM for antagonist activity. Dose-response data, EC₅₀/IC₅₀, from test compound was analyzed with XLfit (IDBS) software.

This assay may be used to determine the IC₅₀ value for adenosine A₃ antagonists according to the invention (see Example 42).

### Example 42

TABLE 1
Selected adenosine A₃ receptor assay antagonist data.

| Compound | IC₅₀ |
|---|---|
| SPN0238 | 173 nM |
| SPN0169 | 154 nM |
| SPN0234 | 8.2 nM |
| SPN0240 | 94 nM |
| SPN0235 | 12 nM |
| SPN0174 | 209 nM |
| SPN0192 | 816 nM |
| SPN0175 | 391 nM |
| SPN0195 | 683 nM |
| SPN0186 | 638 nM |

1. A compound of Formula I having the structure: 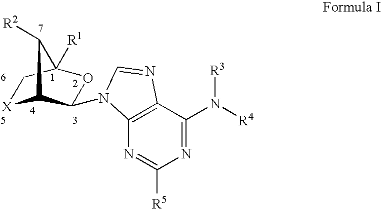
wherein:
X is selected from the group consisting of —O—, —S—, >NH and >NR′, wherein R′ is selected from the group consisting of hydrogen, C₁-C₆ acyl and C₁-C₆ alkyl;
R¹ is selected from the group consisting of R^{a}R^{b}NC(═O)— and HOR^{c}—, wherein R^{a} and R^{b} are independently selected from the group consisting of hydrogen, optionally substituted C₁-C₁₀ alkyl, optionally substituted C₁-C₁₀ acyl, formyl, optionally substituted aryl, and optionally substituted arylcarbonyl; or R^{a} and R^{b} together with the nitrogen atom to which they are attached form an optionally substituted 4- to 8-membered heterocyclic ring; and R^{c} is selected from the group consisting of C₁-C₆ alkylene, C₁-C₆ haloalkylene, and carbonyl;
R² is selected from the group consisting of hydrogen, hydroxy, amino, azido, halo, optionally substituted C₁-C₆ alkyl, optionally substituted C₁-C₆ alkoxy, carboxy, nitrilo, nitro, aryl, thiol, and —Y—CO—R^{d}, wherein Y is selected from the group consisting of —O—, >NH and —S—, and R^{d} is selected from the group consisting of —NH₂, —OH and C₁-C₆ alkyl;
R³ and R⁴ are independently selected from the group consisting of hydrogen, optionally substituted C₁-C₁₀ alkyl, optionally substituted C₁-C₁₀ alkoxycarbonyl, optionally substituted C₁-C₁₀ acyl, formyl, mono- and di(C₁-C₁₀ alkyl)aminocarbonyl, C₁-C₁₀ alkylsulphonyl, C₁-C₁₀ alkylsulphinyl, optionally substituted aryl, optionally substituted arylcarbonyl, optionally substituted heterocyclyl, optionally substituted heterocyclylcarbonyl, optionally substituted heteroaryl, heteroarylcarbonyl; or R³ and R⁴ together with the nitrogen atom to which they are attached form an optionally substituted 4- to 8-membered heterocyclic ring;
R⁵ is selected from the group consisting of hydrogen, halogen, optionally substituted C₁-C₁₀ alkyl, optionally substituted C₂-C₆ alkenyl, optionally substituted C₂-C₆ alkynyl, hydroxyl, optionally substituted C₁-C₁₀ alkoxy, amino, optionally substituted C₁-C₁₀ alkylamino, mercapto, and optionally substituted C₁-C₁₀ alkylthio;
and wherein the stereocenters 1, 3, 4 and 7 may be present in any orientation.

2. The compound according to claim 1, wherein said compound is in the β-D form.

3. The compound according to claim 2, wherein said X is selected from the group consisting of —O— and >NH.

4. The compound according to claim 1, wherein said compound is in the α-L form.

5. The compound according to claim 4, wherein X is selected from the group consisting of >NH and —O—.

6. The compound according to claim 1, wherein said compound is in the xylo form.

7. The compound according to claim 6, wherein X is —O—.

8. The compound according to claim 1, wherein R³ is H.

9. The compound according to claim 1, wherein R⁴ is selected from the group consisting of H and C₁-C₁₀ alkyl.

10. The compound according to claim 1, wherein R⁴ is selected from the group consisting of phenylethyl and benzyl, each of which being optionally substituted in one or more positions with one or more substituents each independently selected from the group consisting of C₁-C_{6 alkyl, amino, halo, C}₁-C₆ haloalkyl, nitro, hydroxy, acetamido, C₁-C₆ alkoxy, and sulfo.

11. The compound according to claim 10, wherein R⁴ is selected from the group consisting of 3-chlorobenzyl, 3-bromobenzyl, and 3-iodobenzyl.

12. The compound according to claim 11, wherein R⁴ is 3-iodobenzyl.

13. The compound according to claim 1, wherein R¹ is selected from the group consisting of —CH₂OH, —CO₂H, and R^{a}R^{b}NC(═O)—, wherein R^{a} and R^{b} are independently selected from the group consisting of H and C₁-C₂ alkyl.

14. The compound according to claim 13, wherein R¹ is —CONHCH₃.

15. The compound according to claim 1, wherein R² is —OH.

16. The compound according to claim 1, wherein R² is —N3.

17. The compound according to claim 1, wherein R² is —NH₂.

18. The compound according to claim 1, wherein R⁵ is selected from the group consisting of Cl, Br and I.

19. The compound according to claim 18, wherein R⁵ is Cl.

20. The compound according to claim 1, where the compound has an IC₅₀ value (adenosine A₃ receptor antagonism) of less than 1 μM.

21. A pharmaceutical composition, comprising a compound according to claim 1 and a pharmaceutically acceptable carrier.

22. A method of treatment or prophylaxis of an adenosine A₃ receptor related disease, comprising administering an effective amount of a compound according to claim 1 to a patient suffering from or at risk of said adenosine A₃ receptor related disease.

23. A method of reducing or alleviating detrimental symptoms associated with chemotherapy comprising: administering an effective amount of a compound according to claim 1 to a patient suffering from or at risk of a disease treatable by chemotherapy, either prior to, during or subsequent to the administration of a chemotherapeutic treatment.

24. A method of enhancing the tolerance of a patient to a chemotherapeutic agent, comprising administering a compound according to claim 1 to a patient suffering from or at risk of a disease treatable by chemotherapy, either prior to, during or subsequent to the administration of chemotherapy treatment.

25. A method for the inhibition of eosinophil or mast cell activation or degranulation, comprising administering a therapeutically effective amount of a compound according to claim 1 to a mammal suffering from a disorder or a disease associated with said activation or degranulation of said eosinophil or mast cells.

26. A method of deactivating an adenosine receptor in a mammal suffering from an adenosine A₃ receptors related disease, comprising administering a therapeutically effective amount of a compound according to claim 1 to said mammal.

27. (canceled)

28. The compound according to claim 1, with the proviso that said compound is other than

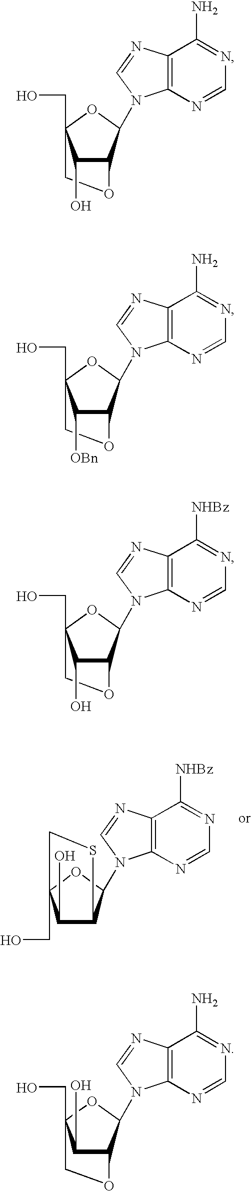

29. The compound according to claim 1, with the following provisos: when R¹ is —CH₂OH and R² is —OH, then (i) R³, R⁴ and R⁵ are not all H, or (ii) X is not selected from the group consisting of —O— and —S—.
